(19) United States
(12) Patent Application Publication
(10) Pub. No.: US 2008/0188524 A1
(43) Pub. Date: Aug. 7, 2008

(54) Methods of treating pain

(76) Inventors: Martin Augustin, Seefeld-Hechendorf (DE); Lutz Zeitlmann, Munchen (DE); Gabriele Stumm, Unterhaching (DE); Michael A. Pleiss, Sunnyvale, CA (US); Philipp Wabnitz, Dusseldrof (DE); Hans Allgeier, Lorrach-Haagen (DE)

Correspondence Address: Olson & Cepuritis, LTD., 20 NORTH WACKER DRIVE, 36TH FLOOR, CHICAGO, IL, 60606, US

(21) Appl. No.: 11/977,275

(22) Filed: Oct. 24, 2007

Related U.S. Application Data
(60) Provisional application No. 60/854,377, filed on Oct. 25, 2006.
(60) Provisional application No. 60/854,134, filed on Oct. 25, 2006.

(51) Int. Cl. A61K 31/4535; A61K 31/425; A61P 25/04

(52) U.S. Cl. 514/326; 514/369

(57) ABSTRACT

A method for the treatment of pain comprises administering a to a subject in need thereof an amount of at least one inhibitor of a cyclin-dependent kinase (CDK). Preferably, the inhibitor of a cyclin-dependent kinase comprises a thiazole of general Formula I, II or III as desribed herein.

### CROSS-REFERENCE TO RELATED APPLICATIONS

This application claims the benefit under 35 U.S.C. §119(e) of U.S. Provisional Application Ser. No. 60/854,377, filed on Oct. 25, 2006, and U.S. Provisional Application Ser. No. 60/854,134, filed on Oct. 25, 2006, each of which is incorporated herein by reference in its entirety.

### FIELD OF THE INVENTION

The present invention relates to inhibitors of cyclin-dependent kinases and therapeutic applications thereof. Furthermore, the invention relates to methods of preventing and/or treating any type of pain comprising the administration of an effective amount of at least one inhibitor of cyclin-dependent kinases.

### BACKGROUND OF THE INVENTION

Cyclin-dependent protein kinases (“CDKs”), constitute a family of well-conserved enzymes that play multiple roles within the cell, such as cell cycle regulation and transcriptional control (Science 1996, Vol. 274:1643-1677; Ann. Rev. Cell Dev. Biol., 1997, 13:261-291.

Some members of the family, such as CDK1, 2, 3, 4, and 6 regulate the transition between different phases of the cell cycle, such as the progression from a quiescent stage in G1 (the gap between mitosis and the onset of DNA replication for a new round of cell division) to S (the period of active DNA synthesis), or the progression from G2 to M phase, in which active mitosis and cell division occur. Other members of this family of proteins, including CDK7, 8, and 9 regulate key points in the transcription cycle, whereas CDK5 plays a role in neuronal and secretory cell function.

CDK complexes are formed through association of a regulatory cyclin subunit (e. g., cyclin A, B1, B2, D1, D2, D3, and E) and a catalytic kinase subunit (e. g., cdc2 (CDK1), CDK2, CDK4, CDK5, and CDK6). As the name implies, the CDKs display an absolute dependence on the cyclin subunit in order to phosphorylate their target substrates, and different kinase/cyclin pairs function to regulate progression through specific portions of the cell cycle.

CDK9 in association with its cyclin partners (cyclin T1, T2a, T2b, or K) constitutes the catalytic component of the positive P-TEFb protein kinase complex that functions during the elongation phase of transcription by phosphorylating the carboxyl-terminal domain (CTD) of the largest subunit of RNA polymerase II. P-TEFb acts in concert with positive transcription factor NfκB as well as negative transcription factors, thus overcoming a block of transcriptional elongation (Liu and Herrmann 2005).

It is known that cell-cycle dysregulation, which is one of the cardinal characteristics of neoplastic cells, is closely associated with genetic alteration and deregulation of CDKs and their regulators, suggesting that inhibitors of CDKs may be useful as therapeutics for proliferative diseases, such as cancer. Thus, small molecule inhibitors targeting CDKs have been the focus of extensive interest in cancer therapy (Current Opinion in Pharmacology, 2003(3): 362-370). The ability of inhibiting cell cycle progression suggests a general role for small molecule inhibitors of CDKs as therapeutics for proliferative diseases, such as cancer. While inhibition of cell cycle-related CDKs is clearly relevant in oncology applications, this may not be the case for the inhibition of RNA polymerase-regulating CDKs. Recently, inhibition of CDK9/cyclin T function was linked to prevention of HIV replication and the discovery of new CDK biology thus continues to open up new therapeutic indications for CDK inhibitors (Sausville, E. A. Trends Molec. Med. 2002, 8, S32-S37) such as, for example, viral infections (WO 02/100401). CDK inhibitors could conceivably also be used to treat other conditions such as immunological diseases and neurodegenerative diseases, amongst others.

More than 50 pharmacological CDK inhibitors have been described, some of which have potent antitumor activity (Current Opinion in Pharmacology, 2003(3): 362-370). A comprehensive review about the known CDK inhibitors may be found in Angew. Chem. Int. Ed. Engl. 2003, 42(19):2122-2138.

The use of 2-anilino-4-phenylpyrimidine derivatives as cyclin-dependent kinase inhibitors for the treatment of e.g. cancer has been reported in WO 2005/012262. Furthermore, 2-pyridinylamino-4-thiazolyl-pyrimidine derivatives for the treatment of cancer etc. have been described in WO 2005/012298. The use of 4,5-dihydro-thiazolo, oxazolo and imidazolo[4,5-h]quinazolin-8-ylamines as protein kinase inhibitors is known from WO 2005/005438. Furthermore, indolinone derivatives and induribin derivatives, which are useful as cyclin-dependent kinase inhibitors, have been disclosed in WO 02/081445 and WO 02/074742. Additionally, CDK inhibitors for various therapeutic applications have been described in WO2005/026129.

Known CDK inhibitors may be classified according to their ability to inhibit CDKs in general or according to their selectivity for a specific CDK. Flavopiridol, for example, acts as a “pan” CDK antagonist and is not particularly selective for a specific CDK (Current Opinion in Pharmacology, 2003(3): 362-370). Purine-based CDK inhibitors, such as olomoucine, roscovitine, purvanolols and CGP74514A are known to exhibit a greater selectivity for CDKs 1, 2 and 5, but show no inhibitory activity against CDKs 4 and 6 (Current Opinion in Pharmacology, 2003(3): 362-370). Furthermore, it has been demonstrated that purine-based CDK inhibitors such as roscovitine can exert anti-apoptotic effects in the nervous system (Pharmacol Ther 2002, 93:135-143) or prevent neuronal death in neurodegenerative diseases, such as Alzheimers's disease (Biochem Biophys Res Commun 2002 (297):1154-1158; Trends Pharmacol Sci 2002 (23):417-425).

Given the tremendous potential of targeting CDKs for the therapy of conditions such as proliferative, immunological, infectious, cardiovascular and neurodegenerative diseases, the development of small molecules as selective inhibitors of particular CDKs constitutes a desirable goal.

The present invention provides novel small molecule inhibitors of cyclin-dependent kinases. Preferably, said small molecule inhibitors show an increased potency to inhibit a particular CDK, in particular CDK9. The small molecule inhibitors of the present invention exert a beneficial effect in the treatment of any type of pain.

Current treatments for pain are only partially effective, and many also cause debilitating or dangerous side effects. For example, many of the traditional analgesics used to treat severe pain induce debilitating side effects such as nausea, dizziness, constipation, respiratory depression, and cognitive dysfunction (Brower, 2000).

Although there is already a broad panel of approved pain medications like non-narcotic analgesics, opioid analgesics, calcium channel blockers, muscle relaxants, and systemic corticosteroids available, said treatments remain merely empirical and, while they may relieve the symptoms of pain, they do not lead to complete relief in most cases. This is also due to fact that the mechanisms underlying the development of the different types of pain are still only poorly understood. Researchers are only just beginning to appreciate the complexity and diversity of the signaling systems used to relay nerve impulses for each type of pain.

Generally, pain is defined as an unpleasant sensory and emotional experience associated with actual or potential tissue damage, or described in terms of such damage, according to the International Association for the Study of Pain (IASP). Specifically, pain may occur as acute or chronic pain.

Acute pain occurs for brief periods of time, typically less than 1 month and is associated with temporary disorders. It is a natural body response to let the host be aware of physiological or biochemical alteration that could result in further damage within a short period of time. It is felt when noxious stimuli activate high threshold mechanical and/or thermal nociceptors in peripheral nerve endings and the evoked action potentials in thinly myelinated (Aδ) and/or unmyelinated (C) afferent fibres reach a conscious brain. Said noxious stimuli may be provided by injury, surgery, illness, trauma or painful medical procedures. Acute pain usually disappears when the underlying cause has been treated or has healed. Unrelieved acute pain, however, may lead to chronic pain problems that may result in long hospital stays, rehospitalizations, visits to outpatient clinics and emergency departments, and increased health care costs.

In contrast to acute pain, chronic pain persists long after the initial injury has healed and often spreads to other parts of the body, with diverse pathological and psychiatric consequences. Chronic somatic pain results from inflammatory responses to trauma in peripheral tissues (e.g., nerve entrapment, surgical procedures, cancer, or arthritis), which leads to oversensitization of nociceptors and intense searing pain responses to normally non-noxious stimuli (hyperalgesia). Chronic pain is continuous and recurrent and its intensity will vary from mild to severe disabling pain that may significantly reduce quality of life.

Chronic pain is currently treated with conventional analgesics such as NSAIDs (Ibuprofen, Naproxen), Cox-2 inhibitors (Celecoxib, Valdecoxib, Rofecoxib) and opiates (codeine, morphin, thebain, papaverin, noscapin). For a significant number of patients however, these drugs provide insufficient pain relief.

Another subtype of pain, inflammatory pain, can occur as acute as well as chronic pain. Resulting injuries of tissue and neurons must not but may develop into long-lasting chronic neuropathic pain effects in succession to such inflammatory events.

Inflammatory pain is mediated by noxious stimuli like e.g. inflammatory mediators (e.g. cytokines, such as TNF α, prostaglandins, substance P, bradykinin, purines, histamine, and serotonine), which are released following tissue injury, disease, or inflammation and other noxious stimuli (e.g. thermal, mechanical, or chemical stimuli). In addition, cytokines and growth factors can influence neuronal phenotype and function (Besson 1999). These mediators are detected by nociceptors (sensory receptors) that are distributed throughout the periphery of the tissue. Said nociceptors are sensitive to noxious stimuli (e.g. mechanical, thermal, or chemical), which would damage tissue if prolonged (Koltzenburg 2000). A special class of so called C-nociceptors represent a class of “silent” nociceptors that do not respond to any level of mechanical or thermal stimuli but are activated in presence of inflammation only.

Current approaches for the treatment of especially inflammatory pain aim at cytokine inhibition (e.g. IL1β) and suppression of pro-inflammatory TNFα. Current approved anticytokine/antiTNFalpha treatments are based on chimeric antibodies such as Infliximab and Etanercept which reduce TNFα circulation in the bloodstream. TNFα is one of the most important inflammatory mediators that induce synthesis of important enzymes such as COX-2, MMP, iNOS, cPLa₂ and others. The main drawbacks of these “biologicals”, however, reside in their immunogenic potential with attendant loss of efficacy and their kinetics that lead to a more or less digital all-or-nothing reduction of circulating TNFalpha. The latter can result in severe immune suppressive side effects.

A distinct form of chronic pain, neuropathic (or neurogenic) pain, arises as a result of peripheral or central nerve dysfunction and includes a variety of conditions that differ in etiology as well as location. Generally, the causes of neuropathic pain are diverse, but share the common symptom of damage to the peripheral nerves or components of central pathways. The causative factors might be metabolic, viral or mechanical nerve lesion. Neuropathic pain is believed to be sustained by aberrant somatosensory processes in the peripheral nervous system, the CNS, or both. Neuropathic pain is not directly linked to stimulation of nociceptors, but instead, is thought to arise e.g. from oversensitization of glutamate receptors on postsynaptic neurons in the gray matter (dorsal horn) of the spinal cord.

Neuropathic pain is associated with conditions such as nerve degeneration in diabetes and postherpetic neuralgia (shingles). Neuropathic pain conditions are the consequence of a number of diseases and conditions, including diabetes, AIDS, multiple sclerosis, stump and phantom pain after amputation, cancer-related neuropathy, post-herpetic neuralgia, traumatic nerve injury, ischemic neuropathy, nerve compression, stroke, spinal cord injury.

Management of neuropathic pain remains a major clinical challenge, partly due to an inadequate understanding of the mechanisms involved in the development and maintenance of neuropathic pain. Many existing analgesics are ineffective in treating neuropathic pain and most of current narcotic and non-narcotic drugs do not control the pain. Current clinical practice includes the use of a number of drug classes for the management of neuropathic pain, for example anticonvulsants, tricyclic antidepressants, and systemic local anaesthetics. However, the usual outcome of such treatment is partial or unsatisfactory pain relief, and in some cases the adverse effects of these drugs outweigh their clinical usefulness. Classic analgesics are widely believed to be poorly effective or ineffective in the treatment of neuropathic pain. Few clinical studies on the use of non steroidal anti-inflammatory drugs (NSAIDs) or opiates in the treatment of neuropathic pain have been conducted, but in those which have, the results appear to indicate that NSAIDs are poorly effective or ineffective and opiates only work at high doses. A review analysing the controlled clinical data for peripheral neuropathic pain (PNP) (Pain, November, 1997 73(2), 123-39) reported that NSAIDs were probably ineffective as analgesics for PNP and that there was no long-term data supporting the analgesic effectiveness of any drug.

Available analgesic drugs often produce insufficient pain relief. Although tricyclic antidepressants and some antiepileptic drugs, for example gabapentin, lamotrigine and carbamazepine, are efficient in some patients, there remains a large unmet need for efficient drugs for the treatment of these conditions.

In conclusion, there is a high unmet need for safe and effective methods of pain treatment, in particular of chronic inflammatory and neuropathic pain.

### SUMMARY OF THE INVENTION

The present invention is directed to inhibitors of cyclin-dependent kinases and to methods and compositions for treating and/or preventing any type of pain comprising: administering an effective amount of at least one inhibitor of a cyclin-dependent kinase (cdk, CDK) to a subject in need thereof. In a preferred embodiment, the inhibitor is selected among compounds according to any one of general Formula I, II or III:

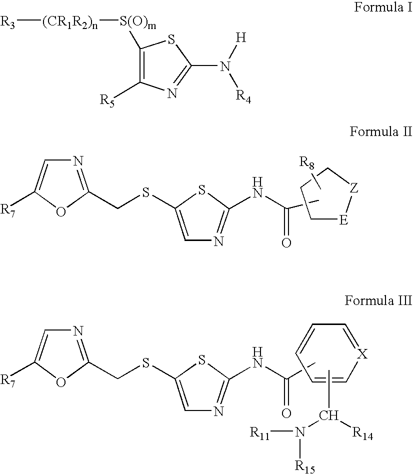

wherein

R₁ and R₂ are independently hydrogen, fluorine or alkyl;

R₃ is aryl or heteroaryl;

R₄ is hydrogen, alkyl, cycloalkyl, aryl, cycloalkylalkyl, arylalkyl, heteroaryl, heteroarylalkyl, heterocycloalkyl, heterocycloalkylalkyl; or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-alkyl, SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkyl-heteroaryl, C(NNO₂)NH-heterocyloalkyl, C(NNO₂)NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHCO-alkyl-aryl, C(NH)NHCO-heteroaryl, C(NH)NHCO-alkyl-heteroaryl, C(NH)NHCO-heterocylcloalkyl, C(NH)NHCO-alkyl-heterocycloaklyl; or C(NOR₆)NH-alkyl, C(NOR₆)NH-cycloalkyl, C(NOR₆)NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;

R₅ is hydrogen or alkyl;

R₆ is hydrogen, alkyl, cycloalkyl, aryl, cycloalkylalkyl, arylalkyl, heteroaryl, heteroarylalkyl, heterocycloalkyl or heterocycloalkylalkyl;

R₇ is alkyl;

R₈ is hydrogen or alkyl;

R₉ and R₁₀ are each independently hydrogen, alkyl, substituted alkyl, cycloalkyl or substituted cycloalkyl;

R₁₁ is hydrogen or alkyl substituted with one or two hydroxyl groups or an amino group (e.g., NR₁₂R₁₃); R₁₅ is hydrogen or alkyl; or R₁₅ and R₁₁ taken together are —(CH₂)_{q}— where q is an integer of 3, 4, 5 or 6;

when present, R₁₂ and R₁₃ are each independently hydrogen, alkyl, substituted alkyl, cycloalkyl or substituted cycloalkyl; or R₁₂ and R₁₃ taken together are —(CH₂)_{q}— where q is an integer of 3, 4, 5 or 6;

R₁₄ is hydrogen or alkyl;

X is CH or N;

Z is NR₉ or CHNR₉R₁₀;

m is 0, 1, or 2;

n is 1, 2, or 3;

E is a covalent bond or (CH₂)ₚ, and

p is 1, 2 or 3;

and the N-oxide derivatives, prodrug derivatives, protected derivatives, individual isomers, mixtures of isomers, enantiomers, diastereomers thereof, and the pharmaceutically acceptable salts and solvates (e. g., hydrates) of such compounds.

In a further embodiment, the invention provides the above-mentioned compounds according to any one of general Formula I, II or III for medical use.

In a further embodiment, the invention provides pharmaceutical compositions containing a compound according to any one of general Formula I, II or III as outlined above, together with a pharmaceutically acceptable carrier.

In a further embodiment, the invention provides the use of a compound according to any of general Formula I, II or III as outlined above for preparing a pharmaceutical composition for treating any type of pain, chronic pain, inflammatory and/or neuropathic pain. In a preferred embodiment, said compound is N-[5-[[[5-(1,1-dimethylethyl)-2-oxazolyl]-methyl]thio]-2-thiazolyl]-4-piperidinecarboxamide.

In a further embodiment, the invention provides a method of treating any type of pain, chronic pain, inflammatory and/or neuropathic pain comprising the administration of an effective amount of at least one of the compounds as mentioned above to a subject in need thereof. In a preferred embodiment, said compound is N-[5-[[[5-(1,1-dimethylethyl)-2-oxazolyl]-methyl]thio]-2-thiazolyl]-4-piperidinecarboxamide.

Furthermore, the present invention relates to a method of inhibiting cdk9, said method comprising administering to a subject in need thereof an effective cdk9 inhibiting amount of at least one compound according to any one of general Formula I, II or III. In a preferred embodiment, said compound is N-[5-[[[5-(1,1-dimethylethyl)-2-oxazolyl]-methyl]thio]-2-thiazolyl]-4-piperidinecarboxamide.

### BRIEF DESCRIPTION OF THE DRAWINGS

FIG. 1 schematically depicts the spared nerve injury model (SNI model, as developed by Decosterd and Woolf, 2000), which is characterized by ligation and section of two branches of the sciatic nerve (tibial and common peroneal nerves) leaving the sural nerve intact.

FIG. 2 schematically depicts the putative role of CDK9 as a target in the development of pain.

FIG. 3 depicts the results of von Frey measurements performed with SNI mice. Von Frey measurements performed at ipsi-lateral (a) and contra-lateral paws (b) of two groups of individual SNI mice (n=7 per group) at day 0 (before surgery), day 19 after surgery (baseline), and day 21 after surgery (pharmacological testing) are shown. Ipsi-lateral baseline values at day 19 represent behavioral measurements without pharmacological treatment. Each column represents an individual mouse.

FIG. 4 depicts the results of ipsi-lateral paw edema measurements performed with carrageenan-treated mice. Within the observation period all animals treated with compound #16 of Table 1 showed reduced paw edema compared to the vehicle control group.

FIG. 5 depicts the results of an in vitro THP-1 assay, indicating that administration of compound #16 of Table 1 reduces LPS-induced cytokine levels. At a concentration of 3 μm, compound #16 of Table 1 reduces the TNFα signal to 50% compared to DMSO (vehicle control).

### DETAILED DESCRIPTION OF THE INVENTION

The present invention is directed to inhibitors of cyclin-dependent kinases and to methods and compositions for treating and/or preventing any type of pain, comprising: administering an effective amount of at least one inhibitor of a cyclin-dependent kinase (cdk, CDK) to a subject in need thereof.

In a preferred embodiment, the inhibitor is selected among compounds according to any one of general Formula I, II or III:

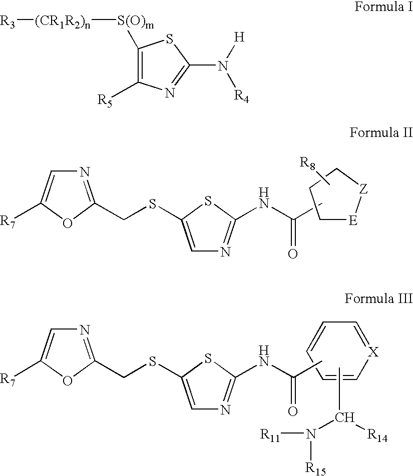

wherein

R₁ and R₂ are independently hydrogen, fluorine or alkyl;

R₃ is aryl or heteroaryl;

R₄ is hydrogen, alkyl, cycloalkyl, aryl, cycloalkylalkyl, arylalkyl, heteroaryl, heteroarylalkyl, heterocycloalkyl, heterocycloalkylalkyl; or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-alkyl, SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, CNNOO₂)NH-alkyl-heteroaryl, C(NNO₂)NH-heterocyloalkyl, C(NNO₂)NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHCO-alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₆)NH-alkyl, C(NOR₆)NH-cycloalkyl, C(NOR₆)NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;

R₅ is hydrogen or alkyl;

R₆ is hydrogen, alkyl, cycloalkyl, aryl, cycloalkylalkyl, arylalkyl, heteroaryl, heteroarylalkyl, heterocycloalkyl or heterocycloalkylalkyl;

R₇ is alkyl;

R₈ is hydrogen or alkyl;

R₉ and R₁₀ are each independently hydrogen, alkyl, substituted alkyl, cycloalkyl or substituted cycloalkyl;

R₁₁ is hydrogen or alkyl substituted with one or two hydroxyl groups or an amino agroup (e.g., NR₁₂R₁₃); R₁₅ is hydrogen or alkyl; or R₁₅ and R₁₁ taken together are —(CH₂)_{q}— where q is an integer of 3, 4, 5 or 6;

when present, R₁₂ and R₁₃ are each independently hydrogen, alkyl, substituted alkyl, cycloalkyl or substituted cycloalkyl, or R₁₂ and R₁₃ taken together are —(CH₂)_{q}— where q is an integer of 3, 4, 5 or 6;

R₁₄ is hydrogen or alkyl;

X is CH or N;

Z is NR₉ or CHNR₉R₁₀;

m is 0, 1, or 2;

n is 1, 2, or 3;

E is a covalent bond or (CH₂)ₚ; and

p is 1, 2 or 3;

and the N-oxide derivatives, prodrug derivatives, protected derivatives, individual isomers, mixtures of isomers, enantiomers, diastereomers thereof; and the pharmaceutically acceptable salts and solvates (e. g., hydrates) of such compounds.

The compounds according to any one of general Formula I, II and III have been described in, e.g. U.S. Pat. No. 6,040,321, U.S. Pat. No. 6,521,759, U.S. Pat. No. 6,515,004 and U.S. Pat. No. 6,214,852, which are incorporated herein in entirety. Methods of manufacturing the compounds according to any one of general Formula I, II and III may be found in the above-referenced U.S. patents.

As used herein, the term “carboxylate anion” refers to a negatively charged group —COO⁻—.

The term “alkyl” or “alk” as used herein refers to a monovalent alkane (hydrocarbon) derived radical containing from 1 to 12 carbon atoms unless otherwise defined. An “alkyl group” is an optionally substituted straight, branched or cyclic saturated hydrocarbon group. When substituted, alkyl groups may be substituted with up to four substituent groups “R” as defined, at any available point of attachment. When the alkyl group is said to be substituted with an alkyl group, this is used interchangeably with “branched alkyl group”. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like. Exemplary substituents may include but are not limited to one or more of the following groups: halo (such as F, Cl, Br, I), haloalkyl (such as CCl₃ or CF₃), alkoxy, alkylthio, hydroxy, carboxy (—COOH), alkyloxycarbonyl (—C(O)R), alkylcarbonyloxy (—OCOR), amino (—NH₂), carbamoyl (—NHCOOR— or —OCONHR—), urea (—NHCONHR—) or thiol (—SH). Alkyl groups as defined may also comprise one or more carbon to carbon double bonds or one or more carbon to carbon triple bonds.

As used herein, the term “alkenyl” refers to a straight, branched or cyclic hydrocarbon radical containing from 2 to 12 carbon atoms and at least one carbon to carbon double bond.

As used herein, the term “alkynyl” refers to a straight, branched or cyclic hydrocarbon radical containing from 2 to 12 carbon atoms and at least one carbon to carbon triple bond.

As used herein, a “cycloalkyl” is a species of alkyl containing from 3 to 15 carbon atoms, without alternating or resonating double bonds between carbon atoms. It may contain from 1 to 4 rings. Exemplary unsubstituted such groups include cyclopropyl, cyclobutyl, cyclopentyl, cyclohexyl, adamantyl, etc. Exemplary substituents include one or more of the following groups: halogen, alkyl, alkoxy, alkyl hydroxy, amino, nitro, cyano, thiol and/or alkylthio.

The terms “alkoxy” or “alkylthio”, as used herein, denote an alkyl group as described above bonded through an oxygen linkage (—O—) or a sulfur linkage (—S—), respectively.

The term “alkyloxycarbonyl”, as used herein, denotes an alkoxy group bonded through a carbonyl group. An alkoxycarbonyl radical is represented by the Formula: —C(O)OR, wherein the R group is a straight or branched C₁₋₆ alkyl group.

As used herein, the term “alkylcarbonyl” refers to an alkyl group bonded through a carbonyl group.

The term “alkylcarbonyloxy” as used herein, denotes an alkylcarbonyl group which is bonded through an oxygen linkage.

The term “arylalkyl”, as used herein, denotes an aromatic ring bonded to an alkyl group as described above.

As used herein, the term “aryl” refers to monocyclic or bicyclic aromatic rings, e.g. phenyl, substituted phenyl and the like, as well as groups which are fused, e.g., napthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Aryl groups may optionally be substituted with one or more groups including, but not limited to halogen, alkyl, alkoxy, hydroxy, carboxy, carbamoyl, alkyloxycarbonyl, nitro, trifluoromethyl, amino, cycloalkyl, cyano, alkyl S(O)ₘ (m=0, 1, 2), or thiol.

The term “heteroaryl” as used herein refers to a monocyclic aromatic hydrocarbon group having 5 or 6 ring atoms, or a bicyclic aromatic group having 8 to 10 atoms, containing at least one heteroatom, O, S, or N, in which a carbon or nitrogen atom is the point of attachment, and in which one or two additional carbon atom(s) is/are optionally replaced by a heteroatom selected from O or S, and in which from 1 to 3 additional carbon atoms are optionally replaced by nitrogen heteroatoms, said heteroaryl group being optionally substituted as described herein. Exemplary heteroaryl groups include the following: thienyl, furyl, pyrrolyl, pyridinyl, imidazolyl, pyrrolidinyl, piperidinyl, thiazolyl, oxazolyl, triazolyl, pyrazolyl, isoxazolyl, isothiazolyl, pyrazinyl, pyridazinyl, pyrimidinal, triazinylazepinyl, indolyl, isoindolyl, quinolinyl, isoquinolinyl, benzothiazolyl, benzoxazolyl, benzimidazolyl, benzoxadiazolyl, benzofurazanyl and tetrahydropyranyl. Exemplary substituents include one or more of the following: halogen, alkyl, alkoxy, hydroxy, carboxy, carbamoyl, alkyloxycarbonyl, trifluoromethyl, cycloalkyl, nitro, cyano, amino, alkylS(O)ₘ (m=0, 1, 2), or thiol.

As used herein, the term “heteroarylium” refers to heteroaryl groups bearing a quaternary nitrogen atom and thus a positive charge.

As used herein, the term “heterocycloalkyl” refers to a cycloalkyl group (nonaromatic) in which one of the carbon atoms in the ring is replaced by a heteroatom selected from O, S or N, and in which up to three additional carbon atoms may be replaced by said heteroatoms.

The term “quaternary nitrogen” as used herein refers to a tetravalent positively charged nitrogen atom including, e.g. the positively charged nitrogen in a tetraalkylammonium group (e.g. tetramethylammonium, N-methylpyridinium), the positively charged nitrogen in protonated ammonium species (e.g. trimethylhydroammonium, N-hydropyridinium), the positively charged nitrogen in amine N-oxides (e.g. N-methyl-morpholine-N-oxide, pyridine-N-oxide), and the positively charged nitrogen in an N-amino-ammonium group (e.g. N-aminopyridinium).

The term “heteroatom” means O, S or N, selected on an independent basis.

The term “halogen” or “halo” refers to chlorine, bromine, fluorine or iodine.

It should be noted that any heteroatom with unsaturated valences is assumed to have the hydrogen atom to saturate the valences.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al., Protective Groups in Organic Synthesis, Wiley, N.Y. (1991).

Suitable examples of salts of the compounds according to the invention with inorganic or organic acids are hydrochloride, hydrobromide, sulfate, phosphate. Salts, which are unsuitable for pharmaceutical uses, but which can be employed, for example, for the isolation or purification of free compounds or their pharmaceutically acceptable salts, are also included.

All stereoisomers of the compounds of the instant invention are contemplated, either in admixture or in pure or substantially pure form. The definition of the compounds according to the invention embraces all possible stereoisomers and their mixtures. In particular, it embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization.

It should be understood that solvates (e.g., hydrates) of the compounds of Formula I or Formula II or Formula III are also within the scope of the present invention. Methods of solvation are generally known in the art. Accordingly, the compounds of the instant invention may be in the free or hydrate form.

A preferred embodiment of the present invention relates to compounds according to any one of general Formula I, II or III:

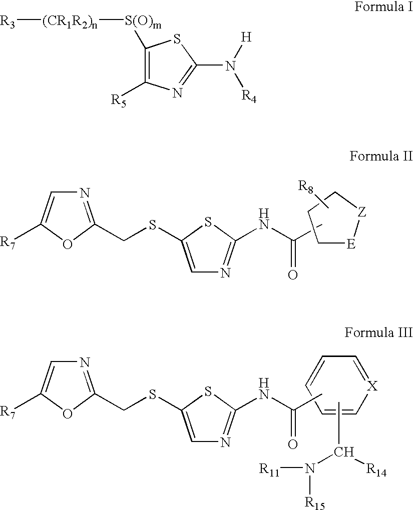

wherein

R₁ and R₂ are independently hydrogen, fluorine or alkyl;

R₃ is aryl or heteroaryl;

R₄ is hydrogen, alkyl, cycloalkyl, aryl, cycloalkylalkyl, arylalkyl, heteroaryl, heteroarylalkyl, heterocycloalkyl, heterocycloalkylalkyl; or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-alkyl, SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)NH-aryl, C(NCN)NH-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkyl-heteroaryl, C(NNO₂)NH-heterocyloalkyl, C(NNO₂)NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHCO-alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₆)NH-alkyl, C(NOR₆)NH-cycloalkyl, C(NOR₆)NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;

R₅ is hydrogen or alkyl;

R₆ is hydrogen, alkyl, cycloalkyl, aryl, cycloalkylalkyl, arylalkyl, heteroaryl, heteroarylalkyl, heterocycloalkyl or heterocycloalkylalkyl;

R₇ is alkyl;

R₈ is hydrogen or alkyl;

R₉ and R₁₀ are each independently hydrogen, alkyl, substituted alkyl, cycloalkyl or substituted cycloalkyl;

R₁₁ is hydrogen or alkyl substituted with one or two hydroxyl groups or an amino agroup (e.g., NR₁₂R₁₃); or R₁₅ and R₁₁ taken together are —(CH₂)_{q}— where q is an integer of 3, 4, 5 or 6;

R₁₂ and R₁₃ are each independently hydrogen, alkyl, substituted alkyl, cycloalkyl or substituted cycloalkyl, or R₁₂ and R₁₃ are taken together with the nitrogen atom to which they are attached to form a 4- to 7-membered heterocyclic ring wherein R₁₂R₁₃ is represented by —(CH₂)_{q}— where q is an integer of 3, 4, 5 or 6;

R₁₄ is hydrogen or alkyl;

R₁₅ is hydrogen or alkyl; and

X is CH or N;

Z is NR₉ or CHNR₉R₁₀;

m is 0, 1, or 2;

n is 1, 2, or 3;

E is a covalent bond or (CH₂)ₚ; and

p is 0, 1, 2 or 3;

and the N-oxide derivatives, prodrug derivatives, protected derivatives, individual isomers, mixtures of isomers, enantiomers, diastereomers thereof; and the pharmaceutically acceptable salts and solvates (e. g., hydrates) of such compounds.

In a preferred embodiment of the compounds according to general Formula I, R₁ and R₂ are independently hydrogen, fluorine or alkyl;

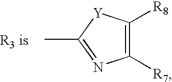

wherein Y is oxygen, sulfur or NR₉;

R₄ is alkyl, cycloalkyl, aryl, cyoloalkylalkyl, arylalkyl, heteroaryl, heteroarylalkyl, heterocycloalkyl, heterocycloalkylalkyl, or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkyl-heteroaryl, C(NON₂)NH-heterocyloalkyl, C(NNO₂) NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHC alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₆)NH-alkyl, C(NOR₆)NH-cycloalkyl, C(NOR₆)NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;

R₅ is hydrogen or alkyl;

R₆ is hydrogen, alkyl, cycloalkyl, aryl, cycloalkylalkyl, arylalkyl, heteroaryl, heteroarylalkyl, heterocycloalkyl or heterocycloalkylalkyl;

R₇ and R₈ are independently hydrogen, alkyl, substituted alkyl, cycloalkyl, aryl, substituted aryl, cycloalkylalkyl, arylalkyl, heteroaryl, substituted heteroaryl, heteroarylalkyl, heterocycloalkyl or heterocycloalkyalkyl;

R₉ is hydrogen, alkyl, cycloalkyl, aryl, alkylcycloalkyl, arylalkyl, heteroaryl, heteroarylalkyl, heterocycloalkyl or heterocycloalkylalkyl;

m is 0, 1 or 2; and

n is 1, 2, or 3.

Particularly preferred compounds according to general Formula I of the invention are those listed in Table 1.

TABLE 1
Compounds according to general Formula I

| # | Compound (according to general Formula I) |
|---|---|
| 1 | N-[5-[[5-Ethyl-2-oxazolyl)methyl]thio]-2-thiazoly]acetamide |
| 2 | N-[5-[[5-Ethyl-2-oxazolyl)methyl]thio]-2-thiazolyl]benzamide |
| 3 | N-[5-[[5-Ethyl-2-oxazolyl)methyl]thio]-2- |
| | thiazolyl]benzenesulfonamide |
| 4 | N-[5-[[(4,5-Dimethyl-2-oxazolyl)methyl]thio]-2- |
| | thiazolyl]acetamide |
| 5 | N-[5-[[(5-t-Butyl-2-oxazolyl)methyl]thio]-2- |
| | thiazolyl]acetamide |
| 6 | N-[5-[[5-t-Butyl-2-oxazolyl)methyl]thio]-2- |
| | thiazolyl]trimethylacetamide |
| 7 | N-[5-[[(4-Ethyl-2-oxazolyl)methyl]thio]-2-thiazolyl]acetamide |
| 8 | N-[5-[[(5-t-Butyl-2-oxazolyl)methyl]thio]-2-thiazolyl]-N′- |
| | cyano-N″-(2,6-difluorophenyl)guanidine |
| 9 | N-[5-[[(5-Isopropyl-2-oxazolyl)fluoromethyl]thio]-2- |
| | thiazolyl]acetamide |
| 10 | N-[5-[[(5-t-Butyl-2-oxazolyl)methyl]thio]-2- |
| | thiazolyl]aminophenyl-4-(2-hydroxyethyl)sulfonamide |
| 11 | N-[5-[[(5-t-Butyl-2-oxazolyl)methyl]thio]-2- |
| | thiazoly]aminophenyl-4-sulfonamide |
| 12 | N-[5-[[(5-t-Butyl-2-oxazolyl)methyl]thio]-2-thiazolyl]-4- |
| | aminopyrimidine |
| 13 | N-[5-[[(5-t-Butyl-2-oxazolyl)methyl]thio]-2-thiazolyl]-3- |
| | (hydroxymethyl)aniline |
| 14 | N-[5-[[(5-t-Butyl-2-oxazolyl)methyl]thio]-2-thiazolyl]-2- |
| | aminopyridine |
| 15 | N-[5-[[(5-t-Butyl-2-oxazolyl)methyl]thio]-2-thiazolyl]-2-[5- |
| | [(((3-hydroxy-2,2- |
| | dimethyl)propyl)amino)methyl]]aminopyridine |
| 16 | N-[5-[[[5-(1,1-dimethylethyl)-2-oxazolyl]-methyl]thio]-2- |
| | thiazolyl]-4-piperidinecarboxamide |

In a preferred embodiment of the compounds according to general Formula II,

R₇ is alkyl;

R₈ is hydrogen or alkyl;

Z is NR₉ or CHNR₉R₁₀;

R₉ and R₁₀ are each independently hydrogen, alkyl, substituted alkyl, cycloalkyl or substituted cycloalkyl; and

p is 0, 1, 2 or 3; and enantiomers, diastereomers and pharmaceutically acceptable salts thereof.

In a particular preferred embodiment of the compounds according to general Formula II,

R₇ is alkyl;

R₈ is hydrogen;

Z is NR₉ or CHNR₉R₁₀;

R₉ and R₁₀ are each independently hydrogen, alkyl, substituted alkyl or cycloalkyl; and

p is 2.

It is also preferred that the compounds have a structure according to general Formula IIa:

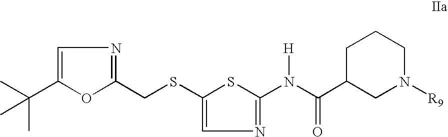

wherein R₉ is hydrogen, alkyl, substituted alkyl or cycloalkyl and enantiomers, diastereomers and pharmaceutically acceptable salts thereof.

It is also preferred that the compounds have a structure according to general Formula IIb:

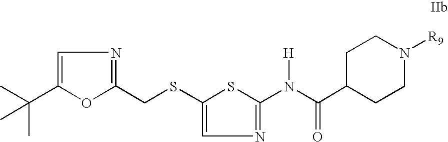

wherein R₉ is alkyl, substituted alkyl or cycloalkyl and enantiomers, diastereomers and pharmaceutically acceptable salts thereof.

It is furthermore preferred that compounds have a structure according to general Formula IIc:

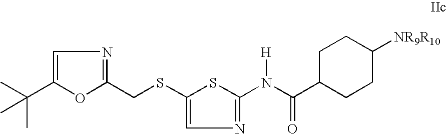

wherein R₉ and R₁₀ are each independently hydrogen, alkyl, substituted alkyl or cycloalkyl with the proviso that both R₉ and R₁₀ cannot be hydrogen and enantiomers, diasteromers and pharmaceutically acceptable salts thereof.

Particularly preferred compounds according to general Formula II of the invention are those listed in Table 2.

TABLE 2
Compounds according to general Formula II

| # | Compound (according to general Formula II) |
|---|---|
| 1 | (±)-N-[5-[[[5-(1,1-dimethylethyl)-2-oxazolyl]methyl]thio]-2- |
| | thiazolyl]-3 Piperidinecarboxamide |
| 2 | (±)-1-(2,3-dihydroxypropyl)-N-[5-[[[5-(1,1-dimethylethyl)-2- |
| | oxazolyl]methyl]thio]-2-thiazolyl]-4-piperidinecarboxamide |
| 3 | N-[5-[[[5-(1,1-dimethylethyl)-2-oxazolyl]methyl]thio]-2- |
| | thiazolyl]-1-(1-methylethyl)-4-piperidinecarboxamide; |
| 4 | 1-cyclopropyl-N-[5-[[[5-(1,1-dimethylethyl)-2- |
| | oxazolyl]methyl]thio]-2-thiazolyl]-4-piperidinecarboxamide; |
| 5 | N-[5-[[[5-(1,1-dimethylethyl)-2-oxazolyl]methyl]thio]-2- |
| | thiazolyl]-1-(2-hydroxyethyl)-4-piperidinecarboxamide; |
| 6 | (R)-N-[5-[[[5-(1,1-dimethylethyl)-2-oxazolyl]methyl]thio]-2- |
| | thiazolyl]-3-piperidinecarboxamide; |
| 7 | (S)-N-[5-[[[5-(1,1-dimethylethyl)-2-oxazolyl]methyl]thio]-2- |
| | thiazolyl]-3-piperidinecarboxamide; |
| 8 | cis-4-amino-N-[5-[[[5-(1,1-dimethylethyl)-2- |
| | oxazolyl]methyl]thio]-2-thiazolyl]cyclohexylcarboxamide; |
| 9 | trans-4-amino-N-[5-[[[5-(1,1-dimethylethyl)-2- |
| | oxazolyl]methyl]thio]-2-thiazolyl]cyclohexylcarboxamide; |

In a preferred embodiment of the compounds according to general Formula III of the present invention,

R₇ is alkyl;

R₈ and R₁₄ are each independently hydrogen or alkyl;

R₁₅ is hydrogen or alkyl, and

R₁₁ is hydrogen or alkyl substituted with one or two hydroxy groups or with a NR₁₂R₁₃ group, or

R₁₅ and R₁₁ are taken together with the nitrogen atom to which they are attached to form a 4- to 7-membered heterocyclic ring wherein R₁₅ R₁₁ is represented by —(CH₂)_{q}— where q is an integer of 3, 4, 5 or 6;

R₁₂ and R₁₃ are each independently hydrogen, alkyl, substituted alkyl, cycloalkyl or substituted cycloalkyl, or R₁₂ and R₁₃ are taken together with the nitrogen atom to which they are attached to form a 4- to 7-membered heterocyclic ring wherein R₁₂R₁₃ is represented by —(CH₂)_{q}— where q is an integer of 3, 4, 5 or 6; and

X is CH or N,

and enantiomers, diastereomers and pharmaceutically acceptable salts thereof.

In a particular preferred embodiment of the compounds according to general Formula III,

R₇ is tert-butyl;

R₈ and R₁₄ are each independently hydrogen or methyl;

R₁₅ is hydrogen, and

R₁₁ is hydrogen, —CH₂C(CH₃)₂CH₂OH, —CH₂CH₂OH, —C(CH₃)₂—CH₂OH, —CH(CH₂OH)₂, —CH₂CH(OH)CH₂OH, —CH(CH₃)CH₂OH or

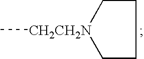

or

R₁₅ and R₁₁ taken together are —(CH₂)₄—; and

X is CH or N.

It is also intended that compounds have a structure according to general Formula IIIa:

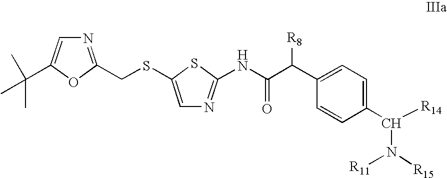

wherein

R₈ and R₁₄ are each independently hydrogen or methyl; and

R₁₅ is hydrogen, and

R₁₁ is hydrogen, —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

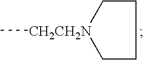

or

R₁₅ and R₁₁ taken together are represented by —(CH₂)₄—.

In a preferred embodiment of the compounds according to general Formula IIIa, R₈ and R₁₄ are each independently hydrogen or methyl; and R₁₅ is hydrogen, and R₁₁ is —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

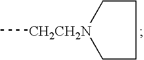

or

R₁₅ and R₁₁ are taken together with the nitrogen atom to which they are attached to form a 5-membered heterocyclic ring where R₁₅R₁₁ is represented by —(CH₂)₄—.

It is also preferred that compounds have the structure according to general Formula IIIb:

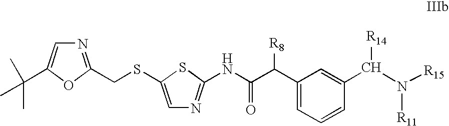

wherein

R₈ and R₁₄ are each independently hydrogen or methyl; and R₁₅ is hydrogen, and R₁₁ is hydrogen, —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

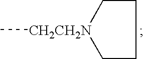

or R₁₅ and R₁₁ taken together are —(CH₂)₄—.

In a preferred embodiment of the compounds according to general Formula IIIb, R₈ and R₁₄ are each independently hydrogen or methyl; and R₁₅ is hydrogen, and R₁₁ is —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

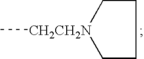

or R₁₅ and R₁₁ taken together are —(CH₂)₄—.

It is furthermore preferred that compounds have the structure according to general Formula IIIc:

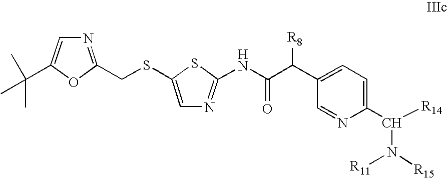

wherein

R₈ and R₁₄ are each independently hydrogen or methyl; and R₁₅ is hydrogen, and R₁₁ is hydrogen, —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

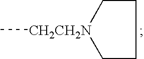

or R₁₅ and R₁₁ are taken together with the nitrogen atom to which they are attached to form a 5-membered heterocyclic ring where R₁₅R₁₁ is represented by —(CH₂)₄—.

In a preferred embodiment of the compounds according to general Formula IIIc, R₈ and R₁₄ are each independently hydrogen or methyl; and R₁₅ is hydrogen, and R₁₁ is —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

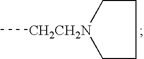

or R₁₅ and R₁₁ are taken together with the nitrogen atom to which they are attached to form a 5-membered heterocyclic ring where R₁₅R₁₁ is represented by —(CH₂)₄—.

Particularly preferred compounds according to general Formula III of the invention are those listed in Table 3.

TABLE 3
Compounds according to general Formula III

| # | Compound (according to general Formula III) |
|---|---|
| 1 | N-[5-[[[5-(1,1-dimethylethyl)-2-oxazolyl]methyl]thio]-2- |
| | thiazolyl]-4-[[[2-hydroxy-1- |
| | (hydroxymethyl)ethyl]amino]methyl]benzeneacetamide |
| 2 | 4-[[(2,3-dihydroxypropyl)amino]methyl]-N-[5-[[[5-(1,1- |
| | dimethylethyl)-2-oxazolyl]methyl]thio]-2- |
| | thiazolyl]benzeneacetamide; |
| 3 | (R)-4-[[(2,3-dihydroxypropyl)amino]methyl]-N-[5-[[[5-(1,1- |
| | dimethylethyl)-2-oxazolyl]methyl]thio]-2- |
| | thiazolyl]benzeneacetamide; |
| 4 | (S)-4-[[(2,3-dihydroxypropyl)amino]methyl]-N-[5-[[[5-(1,1- |
| | dimethylethyl)-2-oxazolyl]methyl]thio]-2- |
| | thiazolyl]benzeneacetamide; |
| 5 | 4-(aminomethyl)-N-[5-[[[5-(1,1-dimethylethyl)-2- |
| | oxazolyl]methyl]thio]-2-thiazolyl]benzeneacetamide; |
| 6 | N-[5-[[[5-(1,1-dimethylethyl)-2-oxazolyl]methyl]thio]-2- |
| | thiazolyl]-4-[[(3-hydroxy-2,2- |
| | dimethylpropyl)amino]methyl]benzeneacetarnide; |
| 7 | N-[5-[[[5-(1,1-dimethylethyl)-2-oxazolyl]methyl]thio]-2- |
| | thiazolyl]-4-(1-pyrrolidinylmethyl)benzeneacetamide; |
| 8 | N-[5-[[[5-(1,1-dimethylethyl)-2-oxazolyl]methyl]thio]-2- |
| | thiazolyl]-4-[[(2-hydroxyethyl)amino]methyl]benzeneacetamide; |
| 9 | N-[5-[[[5-(1,1-dimethylethyl)-2-oxazolyl]methyl]thio]-2- |
| | thiazolyl]-4-[[[2-(1 |
| | pyrrolidinyl)ethyl]amino]methyl]benzeneacetamide; |
| 10 | N-[5-[[[5-(1,1-dimethylethyl)-2-oxazolyl]methyl]thio]-2- |
| | thiazolyl]-4-[[(2-hydroxy-1,1- |
| | dimethylethyl)amiono]methyl]benzeneacetamide; |
| 11 | N-[5-[[[5-(1,1-dimethylethyl)-2-oxazolyl]methyl]thio]-2- |
| | thiazolyl]-3-[[(2-hydroxyethyl)amino]methyl]benzeneacetamide; |
| 12 | N-[5-[[[5-(1,1-dimethylethyl)-2-oxazolyl]methyl]thio]-2- |
| | thiazolyl]-3-[[(3-hydroxy-2,2- |
| | dimethylpropyl)amino]methyl]benzeneacetamide; |
| 13 | 3-(aminomethyl)-N-[5-[[[5-(1,1-dimethylethyl)-2- |
| | oxazolyl]methyl]thio]-2-thiazolyl]benzeneacetamide; |
| 14 | N-[5-[[[5-(1,1-dimethylethyl)-2-oxazolyl]methyl]thio]-2- |
| | thiazolyl]-4-[[(2-hydroxy-1- |
| | methylethyl)amino]methyl]benzeneacetamide; |
| 15 | N-[5-[[[5-(1,1-dimethylethyl)-2-oxazolyl]methyl]thio]-2- |
| | thiazolyl]-4-[1-[(2-hydroxyethyl)amino]ethyl]benzeneacetamide; |
| 16 | N-[5-[[[5-(1,1-dimethylethyl)-2-oxazolyl]methyl]thio]-2- |
| | thiazolyl]-4-[1-[(2-hydroxy-1- |
| | methylethyl)amino]ethyl]benzeneacetamide; |
| 17 | (α)-N-[5-[[[5-(1,1-dimethylethyl)-2-oxazolyl]methyl]thio]-2- |
| | thiazolyl]-4-[[(2-hydroxyethyl)amino]methyl]-α- |
| | methylbenzeneacetamide; |
| 18 | N-[5-[[[5-(1,1-dimethylethyl)-2-oxazolyl]methyl]thio]-2- |
| | thiazolyl]-6-[[(2-hydroxyethyl)amino]methyl]-3- |
| | pyridineacetamide; and |
| 19 | N-[5-[[[5-(1,1-dimethylethyl)-2-oxazolyl]methyl]thio]-2- |
| | thiazolyl]-4-[[(2-hydroxyethyl)amino]methyl]-.alpha.- |
| | methylbenzeneacetamide; |

In a particular preferred embodiment of this invention, the inhibitor of a cdk is selected among any one of the following compounds:
(±)-N-[5-[[[5-(1,1-dimethylethyl)-2-oxazolyl]methyl]thio]-2-thiazolyl]-3piperidinecarboxamide,
(R)—N-[5-[[[5-(1,1-dimethylethyl)-2-oxazolyl]methyl]thio]-2-thiazolyl]-3-piperidinecarboxamide,
(S)—N-[5-[[[5-(1,1-dimethylethyl)-2-oxazolyl]methyl]thio]-2-thiazolyl]-3-piperidinecarboxamide,
cis-4-amino-N-[5-[[[5-(1,1-dimethylethyl)-2-oxazolyl]methyl]thio]-2-thiazolyl]cyclohexylcarboxamide,
trans-4-amino-N-[5-[[[5-(1,1-dimethylethyl)-2-oxazolyl]methyl]thio]-2-thiazolyl]cyclohexylcarboxamide,
N-[5-[[[5-(1,1-Dimethylethyl)-2-oxazolyl]methyl]thio]-2-thiazolyl]-4-[[[2-hydroxy-1-(hydroxymethyl)ethyl]amino]methyl]benzeneacetamide,
N-[5-[[[5-(1,1-Dimethylethyl)-2-oxazolyl]methyl]thio]-2-thiazolyl-4-[[(2-hydroxy-1,1-dimethylethyl)amino]methyl]benzeneacetamide,
4-[[(2,3-Dihydroxypropyl)amino]methyl]-N-[5-[[[5-(1,1-dimethylethyl)-2-oxazolyl]methyl]thio]-2-thiazolyl]benzeneacetamide,
(R)-4-[[(2,3-Dihydroxypropyl)amino]methyl]-N-[5-[[[5-(1,1-dimethylethyl)-2-oxazolyl]methyl]thio]-2-thiazolyl]benzeneacetamide,
(S)-4-[[(2,3-Dihydroxypropyl)amino]methyl]-N-[5-[[[5-(1,1-dimethylethyl)-2-oxazolyl]methyl]thio]-2-thiazolyl]benzeneacetamide,
N-[5-[[[5-(1,1-dimethylethyl)-2-oxazolyl]-methyl]thio]-2-thiazolyl]-4-piperidinecarboxamide,
   and the N-oxide derivatives, prodrug derivatives, protected derivatives, individual isomers and mixtures of isomers, enantiomers and diastereomers thereof; and the pharmaceutically acceptable salts and solvates (e. g., hydrates) thereof.

In a particularly preferred embodiment of this invention, the inhibitor of a cdk is N-[5-[[[5-(1,1-dimethylethyl)-2-oxazolyl]-methyl]thio]-2-thiazolyl]-4-piperidinecarboxamide, as represented by the following structural formula:

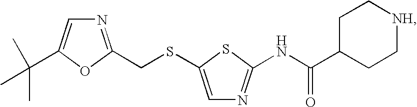

and has the CAS Registry Number 345627-80-7. This compound is also known as BMS-387032 (Sunesis and Bristol-Myers Squibb) and/or SNS-032.

In a preferred embodiment of this invention, the cyclin-dependent kinase inhibitor according to any one of general Formula I, II or III inhibits a CDK selected from the group consisting of CDK1, CDK2, CDK3, CDK4, CDK5, CDK6, CDK7, CDK8, CDK9, CDK10, CDK11, CrkRS (Crk7, CDC2-related protein kinase 7), CDKL1 (cyclin-dependent kinase-like 1); KKIALRE, CDKL2 (cyclin-dependent kinase-like 2), KKIAMRE, CDKL3 (cyclin-dependent kinase-like 3), NKIAMRE, CDKL4, similar to cyclin-dependent kinase-like 1, CDC2L1 (cell division cycle 2-like 1), PITSLRE B, CDC2L1 (cell division cycle 2-like 1), PITSLRE A, CDC2L5 (cell division cycle 2-like 5), PCTK1 (PCTAIRE protein kinase 1), PCTK2 (PCTAIRE protein kinase 2), PCTK3 (PCTAIRE protein kinase 3) or PFTK1 (PFTAIRE protein kinase 1).

The inhibitor may also inhibit more than one cyclin-dependent kinase selected from the above-recited group.

In a particularly preferred embodiment of this invention, the compound according to any one of general Formula I, II or III inhibits CDK9.

In a further embodiment of this invention, the compound according to any one of general Formula I, II or III selectively inhibits one or more CDKs without having a substantial inhibitory effect on other enzymes or proteins.

In a preferred embodiment, such inhibitory compounds display an increased selectivity for a particular CDK. “Increased selectivity” as used herein means that the inhibitory compound is at least 10-100× more selective for a particular CDK selected from the group of CDKs as recited herein, supra. In a preferred embodiment of the present invention, the inhibitory compound is 20-90× more selective for a particular CDK. In a particular preferred embodiment, the inhibitory compound is 30-80× more selective for a particular CDK.

In a particular preferred embodiment, the compound according to any one of general Formula I, II or III displays an increased selectivity for CDK9 than for other CDKs.

As used herein, the term “inhibiting” or “inhibition” refers to the ability of a compound to downregulate, decrease, reduce, suppress, inactivate, or inhibit at least partially the cellular function of a cyclin-dependent kinase, i.e. its activity or the expression of the cyclin-dependent kinase.

Furthermore, the term “cyclin-dependent kinase inhibitor” refers to any compound or group of compounds capable of downregulating, decreasing, suppressing or otherwise regulating the amount and/or activity of a cyclin-dependent kinase. Inhibition of said kinases can be achieved by any of a variety of mechanisms known in the art, including, but not limited to binding directly to the kinase polypeptide, denaturing or otherwise inactivating the kinase, or inhibiting the expression of the gene (e.g., transcription to mRNA, translation to a nascent polypeptide, and/or final polypeptide modifications to a mature protein), which encodes the kinase. Furthermore, a cyclin-dependent kinase inhibitor may also interfere with expression, modification, regulation or activation of a molecule acting downstream of a CDK in a CDK-dependent pathway. Generally, kinase inhibitors may be proteins, polypeptides, nucleic acids, small molecules, or other chemical moieties. Specifically, kinase inhibitors also include monoclonal or polyclonal antibodies directed against cyclin-dependent kinases.

In a preferred embodiment, the cyclin-dependent kinase inhibitor is selected from the compounds as represented by any one of general Formula I, II and III as disclosed herein.

### Therapeutic Use

The compounds according to any one of general Formula I, II or III as provided herein display an unexpected antinociceptive effect.

Thus, in a preferred embodiment, the compounds of any one of general Formula I, II or III may be used in methods and/or pharmaceutical compositions for the treatment of any type of pain, including chronic pain, neuropathic and/or inflammatory pain.

In a particularly preferred embodiment, the compounds of any one of general Formula I, II or III for use in the treatment of any type of pain display an increased selectivity for CDK9 than for other CDKs.

### Pain

Administration of CDK inhibitors according to any one of general Formula I, II or III as disclosed herein to mice suffering from nerve lesion exerts a hypoalgesic effect, in particular in murine models of inflammatory and neuropathic pain.

The discovery that inhibition of a cyclin-dependent kinase is involved in mediating a hypoalgesic effect was unexpected.

Thus, in a preferred embodiment, this invention relates to a method of treating any type of pain comprising administering an effective amount of an inhibitor of cyclin-dependent kinase according to any one of general Formula I, II or III to a subject in need thereof. Specifically, the compounds of any one of general Formula I, II or III may be used for the treatment of chronic, neuropathic and/or inflammatory pain. In a particular preferred embodiment, the compounds of any one of general Formula I, II or III for use in the treatment of any type of pain display an increased selectivity for CDK9 than for other CDKs.

The role of CDK9 in the development of pain is supposed to be based on the following mechanism of action: Both cyclin T1 and CDK9 stimulate the basal promoter activity of TNFα. TNFα is a pro-inflammatory cytokine and pain mediator that controls expression of inflammatory genetic networks. For mediation of cellular TNF receptor responses, the nuclear factor-κB (NFκB) pathway is crucial. TNFα triggers its recruitment to cytokine genes while NfκB interacts with p-TEFb complex for stimulation of gene transcription (Barboric M et al., 2001).

Additionally, it has been shown that CDK9 is a binding partner of TRAF2, a member of the TNFα receptor complex (MacLachlan et al, 1998), while GP130, a subunit of the pro-inflammatory IL6 receptor complex has recently been identified as another potential binding partner of CDK9 (Falco et al, 2002). As a key player in TNFα and interleukin signaling as well as in NfκB mediated expression of several genes (e.g. cytokines as pain mediators), CDK9 can thus be considered as a central target for the treatment of any type of pain, such as inflammatory pain (see FIG. 2).

For the treatment of neuropathic pain, pharmacological action has to take place beyond the blood-brain-barrier (BBB) in the central nervous system (CNS). Microglial cells as the principal immune cells in the CNS, for example, release, upon activation, a variety of noxious factors such as cytokines (TNFα, IL1β, IL6) and other pro-inflammatory molecules (Huwe 2003). Microglia are activated by stimulation of TNFα receptor or Toll-like receptor and signal is mediated via Iκ kinase (IKK) and NfκB leading to transcriptional activation of the cytokines described above. Microglial contribution has been discussed as instrumental in chronic CNS diseases and may contribute to pain perception (Watkins et al, 2003).

Recently it has been shown that the transcription factor NfκB regulates expression of Cyclooxygenase-2 (COX-2) via Interleukin 1β (IL1β) in the spinal cord (Lee et al. 2004). As the major contributor to elevation of spinal prostaglandin E2, the pain mediator COX-2 is already known as a target for a variety of anti-nociceptive/anti-inflamatory drugs. NfκB inhibitors have proven their ability to significantly reduce COX-2 levels and mechanical allodynia as well as thermal hyperalgesia in animal models.

In contrast to COX-2, inhibition of CDK9 action would lead to suppression of a variety of pain mediators instead of just a single one. Thus, anti-nociceptive action of CDK9 inhibitors may be superior compared to e.g. COX-2 inhibitors.

Due to its relevance for NfκB mediated gene transcription, the inhibitory interaction with CDK9 may therefore be a reasonable approach not only for the treatment of acute inflammatory pain, but also for the treatment of chronic pain.

The term “pain” as used herein generally relates to any type of pain and broadly encompasses types of pain such as acute pain, chronic pain, inflammatory and neuropathic pain. In a preferred embodiment of the present invention, “pain” comprises neuropathic pain and associated conditions. The pain may be chronic pain, allodynia (the perception of pain from a normally innocuous stimulus), hyperalgesia (an exaggerated response to any given pain stimulus) and an expansion of the receptive field (i.e. the area that is “painful” when a stimulus is applied), phantom pain or inflammatory pain.

Acute pain types comprise, but are not limited to pain associated with tissue damage, postoperative pain, pain after trauma, pain caused by burns, pain caused by local or systemic infection, visceral pain associated with diseases comprising: pancreatits, intestinal cystitis, dysmenorrhea, Irritable Bowel syndrome, Crohn's disease, ureteral colic and myocardial infarction.

Furthermore, the term “pain” comprises pain associated with CNS disorders comprising: multiple sclerosis, spinal cord injury, traumatic brain injury, Parkinson's disease and stroke. In a preferred embodiment, “pain” relates to chronic pain types comprising headache (for example migraine disorders, episodic and chronic tension-type headache, tension-type like headache, cluster headache, and chronic paroxysmal hemicrania), low back pain, cancer pain, osteoarthritis pain and neuropathic pain, but is not limited thereto.

Inflammatory pain (pain in response to tissue injury and the resulting inflammatory process) as defined herein relates to imflammatory pain associated with diseases comprising connective tissue diseases, rheumatoid arthritis, systemic lupus erythematosus, multiple sclerosis and arthritis, but is not limited thereto.

Neuropathic pain (pain resulting from damage to the peripheral nerves or to the central nervous system itself) includes conditions comprising, but not limited to metabolic neuropathies (e.g., diabetic neuropathy), post-herpetic neuralgia, trigeminal neuralgia, cranial neuralgia, post-stroke neuropathic pain, multiple sclerosis-associated neuropathic pain, HIV/AIDS-associated neuropathic pain, cancer-associated neuropathic pain, carpal tunnel-associated neuropathic pain, spinal cord injury-associated neuropathic pain, complex regional pain syndrome, fibromyalgia-associated neuropathic pain, reflex sympathic dystrophy, phantom limb syndrome or peripheral nerve or spinal cord trauma, nerve transection including surgery, limb amputation and stump pain, pain caused by the side effects of anti-cancer and anti-AIDS therapies, post-surgical neuropathic pain, neuropathy-associated pain such as in idiopathic or post-traumatic neuropathy and mononeuritis, and neuropathic pain caused by connective tissue disease such as rheumatoid arthritis, Wallenberg's syndrome, systemic lupus erythematosus, multiple sclerosis, or polyarteritis nodosa. The neuropathy can be classified as radiculopathy, mononeuropathy, mononeuropathy multiplex, polyneuropathy or plexopathy.

The term “allodynia” denotes pain arising from stimuli which are not normally painful. Allodynic pain may occur other than in the area stimulated.

The term “hyperalgesia” denotes an increased sensitivity to a painful stimulus.

The term “hypoalgesia” denotes a decreased sensitivity to a painful stimulus.

Specifically, the present invention relates to a method for treating the above-referenced types of pain and associated conditions, wherein the term “treating” comprises the prevention, amelioration or treating of pain and associated conditions.

### Pharmaceutical Compositions

Preferred embodiments of the present invention include the administration of compositions comprising at least one cyclin-dependent kinase inhibitor according to any one of general Formula I, II or III as an active ingredient together with at least one pharmaceutically acceptable (i. e. non-toxic) carrier, excipient and/or diluent.

Preferably, the composition comprises at least one cyclin-dependent kinase inhibitor according to any one of general Formula I, II or III as an active ingredient, wherein said at least one cyclin-dependent kinase inhibitor has an increased selectivity for CDK9 than for other CDKs.

Furthermore, the invention also comprises compositions combining at least two inhibitors of CDK and/or pharmaceutically acceptable salts thereof. Said at least two inhibitors may inhibit the same cyclin-dependent kinase or may also inhibit different types of cylin-dependent kinases, e.g. one inhibitor in the composition may inhibit CDK9 while the other inhibitor is capable of inhibiting CDK2, for example.

Having regard to pain treatment, an individual pain medication often provides only partially effective pain alleviation because it interferes with just one pain-transducing pathway out of many. Thus, it is also intended to administer CDK inhibitors according to any one of general Formula I, II or III in combination with a pain-reducing (analgesic) agent that acts at a different point in the pain perception process.

An “analgesic agent” comprises a molecule or combination of molecules that causes a reduction in pain perception. An analgesic agent employs a mechanism of action other than inhibition of CDK.

One class of analgesics, such as nonsteroidal anti-inflammatory drugs (NSAIDs), down-regulates the chemical messengers of the stimuli that are detected by the nociceptors and another class of drugs, such as opioids, alters the processing of nociceptive information in the CNS. Other analgesics are local anesthetics, anticonvulsants and antidepressants such as tricyclic antidepressants. Administering one or more classes of drug in addition to CDK inhibitors can provide more effective amelioration of pain.

Preferred NSAIDs for use in the methods and compositions of the present invention are aspirin, acetaminophen, ibuprofen, and indomethacin. Furthermore, cyclooxygenase-2 (COX-2) inhibitors, such as specific COX-2 inhibitors (e.g. celecoxib, COX189, and rofecoxib) may also be used as an analgesic agent in the methods or compositions of the present invention.

Preferred tricyclic antidepressants are selected from the group consisting of Clomipramine, Amoxapine, Nortriptyline, Amitriptyline, Imipramine, Desipramine, Doxepin, Trimipramine, Protriptylin, and Imipramine pamoate.

Furthermore, the use of anticonvulsants (e.g. gabapentin), GABAB agonists (e.g. L-baclofen), opioids, vanniloid receptor antagonists and cannabinoid (CB) receptor agonists, e.g. CB1 receptor agonists as analgesic is also preferred in the methods and compositions in the present invention.

In preparing cyclin-dependent kinase inhibitor compositions of this invention, one can follow the standard recommendations of well-known pharmaceutical sources such as Remington: The Science and Practice of Pharmacy, 19th ed. (Mack Publishing, 1995).

The pharmaceutical compositions of the present invention can be prepared in a conventional solid or liquid carrier or diluent and a conventional pharmaceutically-made adjuvant at suitable dosage level in a known way. The preferred preparations are adapted for oral application. These administration forms include, for example, pills, tablets, film tablets, coated tablets, capsules, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, wherein said preparations in addition to typical vehicles and/or diluents contain at least one inhibitor according to the present invention and/or a pharmaceutical acceptable salt thereof as active ingredient.

The pharmaceutical compositions according to the present invention containing at least one inhibitor according to the present invention and/or a pharmaceutical acceptable salt thereof as active ingredient will typically be administered together with suitable carrier materials selected with respect to the intended form of administration, i. e. for oral administration in the form of tablets, capsules (either solid filled, semi-solid filled or liquid filled), powders for constitution, gels, elixirs, dispersable granules, syrups, suspensions, and the like, and consistent with conventional pharmaceutical practices. For example, for oral administration in the form of tablets or capsules, the active drug component may be combined with any oral non-toxic pharmaceutically acceptable carrier, preferably with an inert carrier like lactose, starch, sucrose, cellulose, magnesium stearate, dicalcium phosphate, calcium sulfate, talc, mannitol, ethyl alcohol (liquid filled capsules) and the like.

Moreover, suitable binders, lubricants, disintegrating agents and coloring agents may also be incorporated into the tablet or capsule. Powders and tablets may contain about 5 to about 95% by weight of a cyclin-dependent kinase inhibitor according to the Formula I as recited herein or analogues thereof or the respective pharmaceutical active salt as active ingredient.

Suitable binders include starch, gelatin, natural sugars, corn sweeteners, natural and synthetic gums such as acacia, sodium alginate, carboxymethylcellulose, polyethylene glycol and waxes. Among suitable lubricants there may be mentioned boric acid, sodium benzoate, sodium acetate, sodium chloride, and the like.

Suitable disintegrants include starch, methylcellulose, guar gum, and the like.

Sweetening and flavoring agents as well as preservatives may also be included, where appropriate. The disintegrants, diluents, lubricants, binders etc. are discussed in more detail below.

Moreover, the pharmaceutical compositions of the present invention may be formulated in sustained release form to provide the rate controlled release of any one or more of the components or active ingredients to optimise the therapeutic effect(s), e. g. antihistaminic activity and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

Liquid form preparations include solutions, suspensions, and emulsions. As an example, there may be mentioned water or water/propylene glycol solutions for parenteral injections or addition of sweeteners and opacifiers for oral solutions, suspensions, and emulsions. Liquid form preparations may also include solutions for intranasal administration.

Aerosol preparations suitable for inhalation may include solutions and solids in powder form, which may be present in combination with a pharmaceutically acceptable carrier such as an inert, compressed gas, e. g. nitrogen.

For preparing suppositories, a low melting wax, such as a mixture of fatty acid glycerides like cocoa butter is melted first, and the active ingredient is then dispersed homogeneously therein e. g. by stirring. The molten, homogeneous mixture is then poured into conveniently sized moulds, allowed to cool, and thereby solidified.

Also included are solid form preparations, which are intended to be converted, shortly before use, to liquid form preparations for either oral or parenteral administration. Such liquid forms include solutions, suspensions, and emulsions.

The compounds according to the present invention may also be delivered transdermally. The transdermal compositions may have the form of a cream, a lotion, an aerosol and/or an emulsion and may be included in a transdermal patch of the matrix or reservoir type as is known in the art for this purpose.

The term capsule as recited herein refers to a specific container or enclosure made e. g. of methylcellulose, polyvinyl alcohols, or denatured gelatins or starch for holding or containing compositions comprising the active ingredient(s). Capsules with hard shells are typically made of blended or relatively high gel strength gelatins from bones or pork skin. The capsule itself may contain small amounts of dyes, opaquing agents, plasticisers and/or preservatives. Under tablet a compressed or moulded solid dosage form is understood which comprises the active ingredients with suitable diluents. The tablet may be prepared by compression of mixtures or granulations obtained by wet granulation, dry granulation, or by compaction well known to a person of ordinary skill in the art.

Oral gels refer to the active ingredients dispersed or solubilised in a hydrophilic semi-solid matrix.

Powders for constitution refer to powder blends containing the active ingredients and suitable diluents which can be suspended e. g. in water or in juice.

Suitable diluents are substances that usually make up the major portion of the composition or dosage form. Suitable diluents include sugars such as lactose, sucrose, mannitol, and sorbitol, starches derived from wheat, corn rice, and potato, and celluloses such as microcrystalline cellulose. The amount of diluent in the composition can range from about 5 to about 95% by weight of the total composition, preferably from about 25 to about 75% by weight, and more preferably from about 30 to about 60% by weight.

The term disintegrants refers to materials added to the composition to support disintegration and release of the pharmaceutically active ingredients of a medicament. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked microcrystalline celluloses such as sodiumcroscaramellose, alginates such as alginic acid and sodium alginate, clays such as bentonites, and effervescent mixtures. The amount of disintegrant in the composition may range from about 2 to about 20% by weight of the composition, more preferably from about 5 to about 10% by weight.

Binders are substances which bind or “glue” together powder particles and make them cohesive by forming granules, thus serving as the “adhesive” in the Formulation. Binders add cohesive strength already available in the diluent or bulking agent. Suitable binders include sugars such as sucrose, starches derived from wheat corn rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethylcellulose, polyvinylpyrrolidone, and inorganic compounds such as magnesium aluminum silicate. The amount of binder in the composition may range from about 2 to about 20% by weight of the composition, preferably from about 3 to about 10% by weight, and more preferably from about 3 to about 6% by weight.

Lubricants refer to a class of substances which are added to the dosage form to enable the tablet granules etc. after being compressed to release from the mould or die by reducing friction or wear. Suitable lubricants include metallic stearates such as magnesium stearate, calcium stearate, or potassium stearate, stearic acid, high melting point waxes, and other water soluble lubricants such as sodium chloride, sodium benzoate, sodium acetate, sodium oleate, polyethylene glycols and D,L-leucine. Lubricants are usually added at the very last step before compression, since they must be present at the surface of the granules. The amount of lubricant in the composition may range from about 0.2 to about 5% by weight of the composition, preferably from about 0.5 to about 2% by weight, and more preferably from about 0.3 to about 1.5% by weight of the composition.

Glidents are materials that prevent baking of the components of the pharmaceutical composition together and improve the flow characteristics of granulate so that flow is smooth and uniform. Suitable glidents include silicon dioxide and talc.

The amount of glident in the composition may range from about 0.1 to about 5% by weight of the final composition, preferably from about 0.5 to about 2% by weight.

Coloring agents are excipients that provide coloration to the composition or the dosage form. Such excipients can include food grade dyes adsorbed onto a suitable adsorbent such as clay or aluminum oxide. The amount of the coloring agent may vary from about 0.1 to about 5% by weight of the composition, preferably from about 0.1 to about 1% by weight.

The present invention relates to the administration of compositions containing as active ingredient a cyclin-dependent kinase inhibitor according to any one of general Formula I, II and III to a subject in need thereof for the treatment of any type of pain.

“A subject in need thereof” comprises an animal, preferably a mammal, and most preferably a human, expected to experience any type of pain in the near future or which has ongoing experience of said conditions. For example, such animal or human may have an ongoing condition that is causing pain currently and is likely to continue to cause pain, or the animal or human has been, is or will be enduring a procedure or event that usually has painful consequences. Chronic painful conditions such as diabetic neuropathic hyperalgesia and collagen vascular diseases are examples of the first type; dental work, particularly in an area of inflammation or nerve damage, and toxin exposure (including exposure to chemotherapeutic agents) are examples of the latter type.

In order to achieve the desired therapeutic effect, the respective cyclin-dependent kinase inhibitor has to be administered in a therapeutically effective amount.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated. In the context of the present invention, a therapeutically effective amount comprises, e.g., an amount that reduces pain, in particular inflammatory or neuropathic pain. Specifically, a therapeutically effective amount denotes an amount which exerts a hypoalgesic effect in the subject to be treated.

Such effective amount will vary from subject to subject depending on the subject's normal sensitivity to, e.g., pain, its height, weight, age, and health, the source of the pain, the mode of administering the inhibitor of CDKs, the particular inhibitor administered, and other factors. As a result, it is advisable to empirically determine an effective amount for a particular subject under a particular set of circumstances.

### Salts/Esters

The compounds of Formula I, II or III can be present as salts or esters, in particular pharmaceutically acceptable salts or esters. A review of suitable pharmaceutical salts may be found in Berge et al, J Pharm Sci, 66,1-19 (1977). Salts are formed, for example with strong inorganic acids such as mineral acids, e. g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl-or aryl-sulfonic acids which are unsubstituted or substituted (for example, by a halogen) such as methane- or p-toluene sulfonic acid.

In a preferred embodiment, a pharmaceutically acceptable salt is selected from the group consisting of hydrochloride, dihydrochloride, sulfate, trifluoroacetate, mixture of trifluoroacetate and hydrochloride, tartrate, fumarate, succinate, maleate, citrate, methanesulfonate, bromate and iodate salts.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e. g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C1-C4)-alkyl-or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e. g. by a halogen).

### Enantiomers/Tautomers

The present invention also comprises all enantiomers and tautomers of the compounds of Formula I, II or III. The corresponding enantiomers and/or tautomers may be isolated/prepared by methods known in the art.

### Stereo and Geometric Isomers

Some of the compounds of the invention may exist as stereoisomers and/or geometric isomers, e.g. they may possess one or more asymmetric and/or geometric centres and thus, may exist in two or more stereoisomeric and/or geometric forms. The present invention contemplates the use of all the individual stereoisomers and geometric isomers of those inhibitor agents, and mixtures thereof.

### Solvates

The present invention also includes the use of solvate forms of the compounds of the present invention.

Polymorphs

The invention furthermore relates to the compounds of the present invention in their various crystalline forms, polymorphic forms and (an) hydrous forms. It is well established that chemical compounds may be isolated in any of such forms by slightly varying the method of purification and or isolation form the solvents used in the synthetic preparation of such compounds.

### Prodrugs

The invention further includes the compounds of the present invention in prodrug form. Such prodrugs are generally compounds of Formula I, II or III wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo.

Examples of such modifications include ester (for example, any of those described above), wherein the reversion may be carried out be an esterase etc. Other such systems will be well known to those skilled in the art.

The compounds according to any one of general Formula I, II or III can be prepared by any conventional means, known to the skilled artisan. Suitable processes for synthesizing these compounds are provided in U.S. Pat. No. 6,040,321, U.S. Pat. No. 6,521,759, U.S. Pat. No. 6,515,004 and U.S. Pat. No. 6,214,852, which are incorporated herein in entirety. A suitable process for synthesizing N-[5-[[[5-(1,1-dimethylethyl)-2-oxazolyl]-methyl]thio]-2-thiazolyl]-4-piperidinecarboxamide is described in, e.g., WO2002/010162, which is incorporated herein in entirety.

The invention is further illustrated by the following non-limiting examples.

### EXAMPLES

### Example 1

### Behavioral Animal Models For the Analysis of Inflammatory and Neuropathic Pain

Several animal models for the analysis of inflammatory and neuropathic pain are known. Said models share the common feature that after e.g., induction of a nerve lesion (e.g., spared nerve injury, SNI) or after exposing experimental animals to a noxious stimulus (e.g., injection of formalin or carrageenan), the signs of pain as induced by said interventions are measured by quantifiable behavioral components such as, e.g., paw withdrawal threshold to mechanical stimulation with von Frey hairs (or to thermal stimulation using a laser source or licking behaviour). These reactions are interpreted as being equivalent to mechanical and thermal allodynia (hypersensitivity to mechanical stimuli) or hyperalgesia in humans.

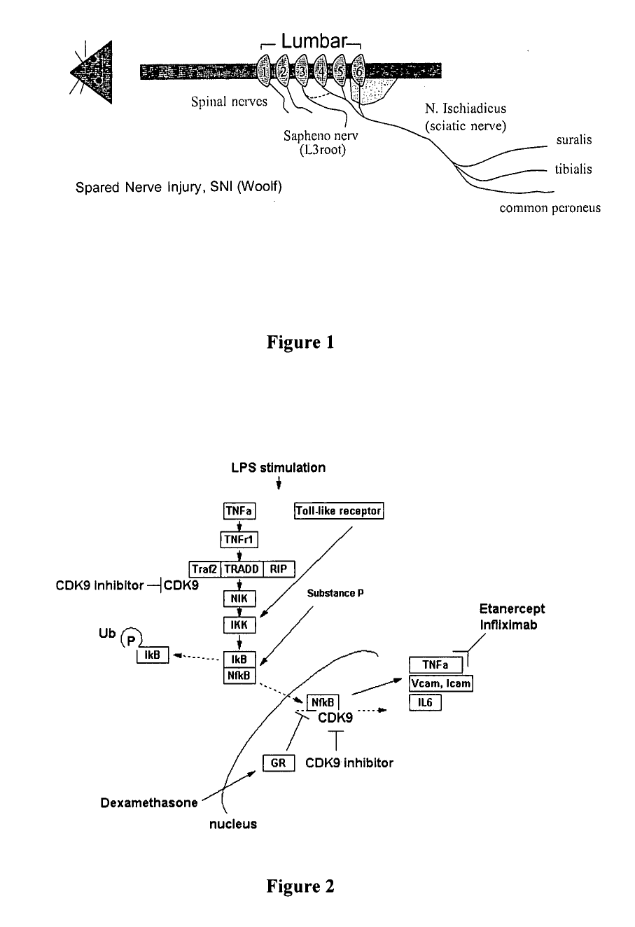

The spared nerve injury model (SNI model, as developed by Decosterd and Woolf (2000), see FIG. 1) is characterized by the induction of clinically relevant nerve lesions and after surgical intervention, subsequent behavioral experiments (e.g., von Frey Assay). Said model constitutes a common nerve injury model which consists of ligation and section of two branches of the sciatic nerve (namely tibial and common peroneal nerves) leaving the sural nerve intact. The SNI model results in early (less than 24 hours), prolonged and substantial changes in mechanical and cold sensitivity that closely mimic the features of clinical neuropathic pain. Animals with these types of nerve injury have been shown to develop abnormal pain sensations and hypersensitivity to mechanical stimuli (allodynia) similar to those reported by neuropathic pain patients.

Alternatively, the formalin assay in mice is a valid and reliable behavioral model of nociception in inflammatory and neuropathic pain. It is sensitive to various classes of analgesic drugs (Hunskaar S, Hole K, Pain. July 1987; 30(1):103-14.) The noxious stimulus consists of an injection of 10 μl diluted formalin (2% in saline) under the skin of the dorsal surface of the left hindpaw (subcutaneous or interplantar into the left hindpaw). The response is licking and flinching of the injected paw.

For the carrageenan assay a subcutaneous injection of 25 μl of 1% carrageenan (in saline) into a single hind paw (ipsi-lateral paw) of mice is applied. Subsequent inflammation results in long lasting swelling and hypersensitivity (against mechanical and thermal stimuli) of the paw. The carrageenan assay is a standard laboratory assay used to predict anti-inflammatory activity of test compounds. Paw edema measurements and Hargreaves Assay (withdrawal of paws due to thermal stimulation via a light source) are used for read out.

Regarding the present invention, the effect of administration of cyclin-dependent kinase (CDK)-inhibiting compounds according to any one of general Formula I, II or III as disclosed herein on the development of inflammatory and neuropathic pain is assayed in a SNI model, in a carrageenan and in a formalin assay. The experimental procedure and results are described in detail below.

### Example 2

### Spared Nerve Injury (SNI)—Model of Chronic Neuropathic Pain

As outlined above, the spared nerve injury (SNI) model (see FIG. 1) involves a lesion of two of the three terminal branches of the sciatic nerve (tibial and common peroneal nerves) of experimental animals, leaving the sural nerve intact. SNI results in mechanical and thermal allodynia in the non-injured sural nerve skin territory (Decosterd and Woolf, Pain 2000; 87:149-158. (2) Tsujino et al., Mol. Cel. Neurosci. 2000; 15:170-182).

### 1. Induction of Spared Nerve Injury (Nerve Lesion) in Wildtype Mice

Wildtype mice (strain C3HeB/FeJ) (age, sex and weight matched) are anesthetized with Hypnorm (0.315 mg/ml fentanyl citrate +10 mg/ml fluanisone; Janssen)/Hypnovel (5 mg/ml midazolam; Roche Applied Sciences)/water at a ratio of 1:1:2 at 4 μl/g prior to surgical preparation.

Subsequently, an incision is made under aseptic precautions in the ipsi-lateral right hind leg of all mice just above the level of the knee, exposing the three terminal branches of the sciatic nerve: the common peroneal, tibial, and sural nerves. The common peroneal and tibial nerves are ligated tightly with 7/0 silk and sectioned distal to the ligation removing 2 mm of distal nerve stump. The sural branch remains untouched during the procedure (denoted herein “SNI ipsi”). The overlying muscle and skin is sutured, and the animals are allowed to recover and to permit wound healing. In the same mice the sciatic nerve branches of the contra-lateral left hind leg are exposed but not lesioned (denoted herein “SNI contra-lateral”). Mice that underwent spared nerve injury are hereinafter denoted “SNI mice”.

### 2. Administration of CDK-Inhibiting Compounds to SNI Mice

After recovery from surgery and wound healing, SNI mice receive per oral (p.o.) injections of CDK-inhibiting compounds.

In this example, N-[5-[[[5-(1,1-dimethylethyl)-2-oxazolyl]-methyl]thio]-2-thiazolyl]-4-piperidinecarboxamide (hereinafter denoted as “compound #16 of Table 1”) was administered.

30 mg/kg of compound #16 of Table 1 were administered in 200 μl of 10% DMSO, 5% Tween 80, 4% Tris 1M pH8, 81% PBS via i.p. application 30 min prior to von Frey measurements (mechanical allodynia). As a negative control, the same amount (200 μl ) of 10% DMSO, 5% Tween 80, 4% Tris 1M pH8, 81% PBS was administered via i.p. application 30 min prior to von Frey measurements.

Injection of inhibitor or vehicle, and subsequent measurements of paw withdrawal threshold to mechanical stimulation in von Frey assays were performed at day 21 post SNI. Reflex nociceptive responses to mechanical stimulation were measured in a von Frey assay 30 min after each injection.

### 3. Behavioral Testing of SNI Mice After Administration of CDK-Inhibiting Compounds (von Frey Assay)

Mice that underwent SNI and subsequent administration of the compounds of the present invention are tested for signs of mechanical allodynia post nerve injury and post administration in a von Frey assay (Decosterd and Woolf, Pain 2000; 87:149-158). This assay determines the mechanical threshold upon which a stimulus, which normally is not painful, is recognized by an animal as uncomfortable or painful. SNI ipsi and SNI contra baselines, respectively, are established.

Mechanical thresholds of SNI mice are quantified using the up-down method based on Chaplan et al. (1994) and Malmberg and Basbaum (1998).

Mice are placed in plexiglass cylinders of about 9.5 cm in diameter, 14 cm high with four vent holes toward the top and a plexiglass lid. The cylinders are placed on an elevated mesh surface (7×7 mm squares). Prior to the day of testing, the mice are acclimated to the testing cylinders for 1-2 hours. On the day of testing the mice are acclimated to the cylinders for about an hour, wherein the acclimation time depends on factors such as the strain of the mouse and the number of times they have been tested previously. In general, testing may begin once the mice are calm and stop exploring the new environment.

For testing mice, filaments 2.44, 2.83, 3.22, 3.61, 3.84, 4.08, and 4.31 (force range=0.04 to 2.0 g) are used. The 3.61 mN filament is applied first. Said filament is gently applied to the plantar surface of one paw, allowed to bend, and held in position for 2-4 seconds. Whenever a positive response to the stimulus (flexion reaction) occurs the next weaker von Frey hair is applied; whenever a negative response (no reaction) occurs the next stronger force is applied. The test is continued until the response to 4 more stimuli after the first change in response has been obtained. The highest force tested is 4.31. The cut-off threshold is 2 g.

The series of scores (i.e, “flexion reaction” and “no reaction” ) and the force of the last filament applied are used to determine the mechanical threshold as described in Chaplan et al., Journal of Neuroscience Methods. 53(1):55-63, July 1994. The threshold determined is that to which the animal would be expected to respond to 50% of the time. Mice are sacrificed after von Frey measurements were accomplished.

### 4. Effects of Administration of CDK-Inhibiting Compounds on the Development of Neuropathic Pain

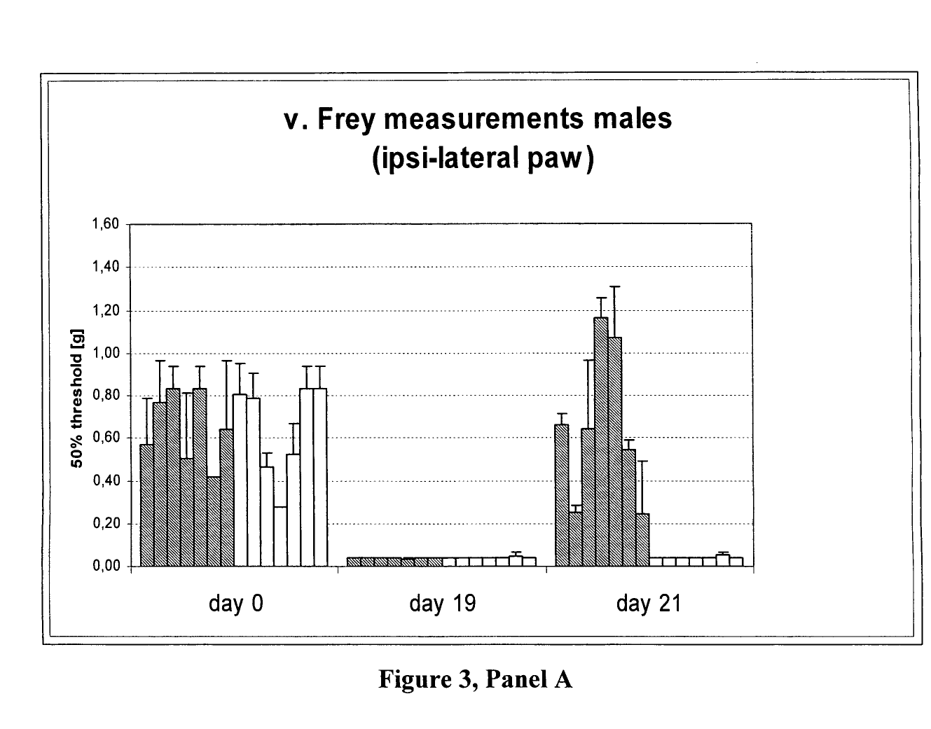

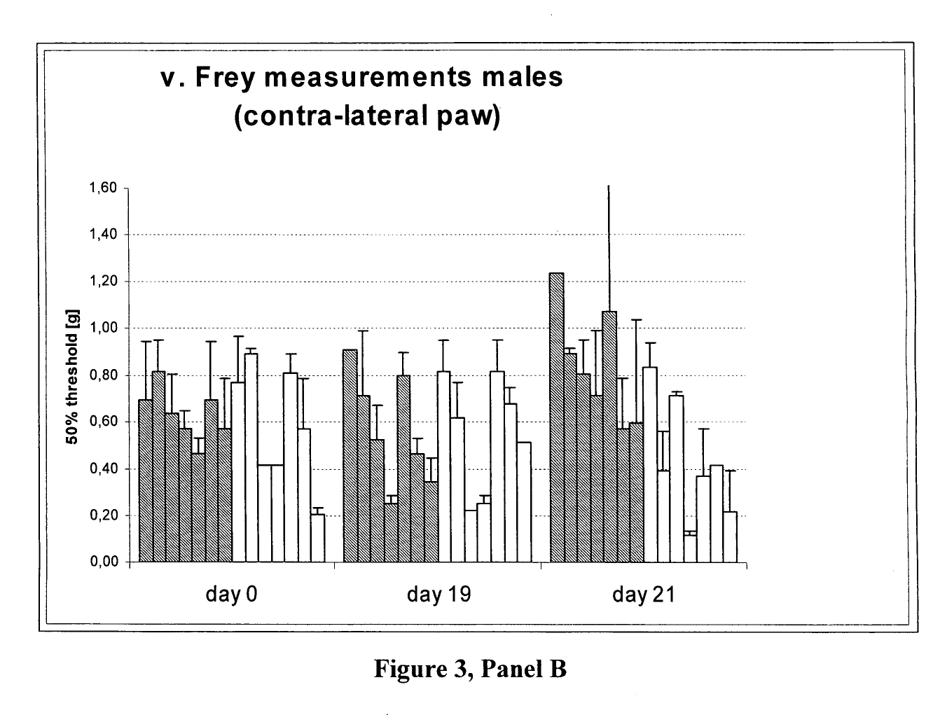

Compound #16 of Table 1 was administered to SNI mice as described above. Von Frey measurements were performed as described above. Compound #16 of Table 1 had a hypoalgesic effect on SNI mice. The results of administration of compound #16 of Table 1 on SNI male mice are shown in FIG. 3.

FIG. 3 depicts the results of von Frey measurements performed with SNI mice. Von Frey measurements performed at ipsi-lateral (a) and contra-lateral paws (b) of two groups of individual SNI mice (n=7 per group) during a time course at day 0 (before surgery), day 19 after surgery (baseline), and day 21 after surgery (pharmacological testing) are shown. Ipsi-lateral baseline values at day 19 represent behavioral measurements without pharmacological treatment. Each column represents an individual mouse. Pharmacological testing of compound #16 of Table 1 and vehicle control was performed 30 minutes prior to behavioural testing with 30 mg/kg compound #16 of Table 1 (group 1, hatched) or vehicle (group 2 solid white), respectively. Ipsi-lateral measurements at day 19 displayed for all animals in both groups high allodynia (=low threshold) in response to a mechanical stimulus. At day 21, i.p. treatment with 30 mg/kg compound #16 of Table 1 resulted in significant amelioration of allodynia. All animals treated with compound #16 of Table 1 displayed a significant increase of threshold values indicating reduced sensitivity to mechanical stimuli (reduced allodynia). In comparison, animals treated with vehicle alone displayed low thresholds comparable to day 19 baseline values indicating high allodynia. The contra-lateral paws of all individuals where unaffected by the surgery as well as by the pharmacological treatment. These findings signify that compound #16 of Table 1 is effective as a hypoalgesic drug in models of chronic neuropathic pain.

### Example 3

### Formalin Assay—Model of Inflammatory Processes/Inflammatory and Chronic Neuropathic Pain

The formalin assay in mice is a valid and reliable behavioral model of nociception and is sensitive to various classes of analgesic drugs (Hunskaar S, Hole K, Pain. July 1987; 30(1):103-14.) The noxious stimulus is an injection of 10 μl diluted formalin (2% in saline) subcutaneous or intraplantar into the left hind paw. The response is licking and flinching of the injected paw. The response shows two phases, which reflect different parts of the inflammatory process (Abbott et al 1995), an early/acute phase 0-5 min post-injection, and a late/chronic phase 5-30 min post-injection. The following protocol describes one possible way to conduct the experiment:

### 1. Injection of Formalin and Administration of CDK-Inhibiting Compound

Age, sex and weight matched wildtype mice (C3HeB/FeJ) are used in this assay. Prior to formalin injection the animals are randomly subdivided into experimental groups of 10 animals each. Thirty minutes prior to formalin injection, a suitable dose of a CDK inhibitor dissolved in (400 μl) of 2% Hydroxprolylcellulose; 0.25% Lactic Acid (85% solution) can be administered by i.p. injection. Similarly, Iκ Kinase (IKK) inhibitor (30 mg/kg) in (400 μl) of 2% Hydroxprolylcellulose; 0.25% Lactic Acid (85% solution) (positive control), or vehicle alone (400 μl) of 2% Hydroxprolylcellulose; 0.25% Lactic Acid (85% solution) (negative control) can be administered by i.p. injection 30 min before formalin injection.

For formalin injection the mouse is held with a paper towel, in order to avoid disturbance of the injection by movements. The injected hind paw is held between thumb and forefinger and 10 μl of Formalin (2%) is injected subcutaneously (s.c.) between the two front tori into the plantar hind paw using a Hamilton syringe. The behavior of the formalin- and inhibitor-treated mice is analyzed as described below.

### 2. Behavioral Analysis of Mice After Injection of Formalin and Administration of CDK-Inhibiting Compound

The behaviour of the formalin-treated mice, i.e. licking and flinching, is monitored by an automated tracking system (Ethovision 3.0 Color Pro, Noldus, Wageningen, Netherlands) over a defined period of time: measurement is initiated 5 min after formalin injection and terminated 30 min after formalin injection. This time frame covers phase II of formalin-induced nociception (pain), which is hyperalgesia.

Two different fluorescent dyes are used for topically marking the injected hind paw (yellow dye) (Lumogenyellow; BASF Pigment, Cologne, Germany) and the contralateral paw (blue dye) (Lumogenviolet; Kremer Pigmente, Aichstetten, Germany) respectively. To determine licking behaviour, mice are monitored with a CCD camera. After monitoring and recording, the video is analyzed using the EthoVision software (Ethovision 3.0 Color Pro, Noldus, Wageningen, Netherlands) or by manual analysis. Fluorescent dot sizes and fluorescence intensities are measured and reduction of fluorescent dot size through licking and biting is calculated. The overall licking time intensity is automatically calculated by comparison of dot size reduction of treated versus untreated paws.

As another variant of assay read out the licking behaviour of the individual animals is tracked manually based on video files. Licking times are recorded over 30 minutes after formalin injection and subdivided for three different licking zones (dorsum, plantar, toes). Overall licking times can be calculated for each animal as well as each experimental group and be used as a parameter for determination of compound efficacy.

As a result it is found that mice receiving vehicle treatment prior to formalin injection (negative control) display a prolonged licking time and a significant reduction of fluorescent dot size at the formalin-treated paw.

In contrast, a reduction in licking time and in consequence no significant reduction of fluorescent dot size of the formalin-treated paw can be observed in test compound/formalin-treated mice. The same effect, i.e. a reduction in licking time and a minor change in fluorescent dot size, is observed in control mice treated with Ikappa kinase inhibitor (IKK; for function of IKK see FIG. 2, positive control).

This observation is indicative for reduced inflammatory/chronic inflammatory pain perception in CDK9 inhibitor-treated mice and for a hypoalgesic effect of the tested compound.

### Example 4

### Carrageenan Assay in Mice—Model of Inflammation and Inflammatory Pain

The model of carrageenan induced paw edema is a standard laboratory assay used to predict anti-inflammatory activity and reduction of inflammation-induced pain perception of respective compounds. The following protocol describes one possible way to conduct the experiment.

The basic measurement constitutes in the measurement of edema and mechanical as well as thermal hypersensitivity in response to irritants, such as carrageenan.

Inflammation and resulting inflammatory pain is induced by subcutaneous injection of 25 μl of 1% carrageenan (in saline) into mice hind paw (ipsi-lateral paw). Each group of 10 mice receives administration of a compound according to any one of general Formula I, II or III, 30 mg/kg body weight, vehicle ((400 μl) of 2% Hydroxprolylcellulose; 0.25% Lactic Acid (85% solution)) and saline (physiol. NaCl) by i.p. injection 30 min prior to carrageenan injection. Contra-lateral paws do not receive carrageenan injection.

### Effects of Administration of a CDK-Inhibiting Compound on Carrageenan-Treated Mice

Paw edema induced by carrageenan injection are detected by increased paw size measured from dorsal to plantar at the metatarsus region of the injected (ipsi-lateral) paws. Sizes of ipsi- and contra-lateral paws serve as surrogate markers for inflammation and are measured at several time points after carrageenan injection: before injection (−1), 2 h (2),3 h (3) 4 h (4), 5 h (5), 6 h (6), 24 h (24) after injection.

The paw size of all mice may increase, e.g., by 2 to 3 mm (+10%) within the first hour after carrageenan injection, independent of the type of treatment substance injected 30 minutes prior to carrageenan. During the time course, mice which received treatment with a CDK-inhibiting compound prior to carrageenan injection may display a reduction of the edema until 24 h after carrageenan injection: the increase in paw size could drop e.g. from 10% down to 8%. In contrast, the paw size of the control mice could increase by 30% in average at this time point. After 24 h post carrageenan injection, the size of all paws treated with carrageenan may increase to reach its maximum at 96 h after injection.

As a read-out of the carrageenan assay, a Hargreaves Assay may be performed, wherein said assay allows the measuring of thermal sensitivity to radiant heat. The Hargreaves assay (Hargreaves et al., 1988) measures nociceptive sensitivity in a freely moving animal by focusing a radiant heat source on the plantar surface of an animal's hindpaw as it stands in a plexiglass chamber. Specifically, the lower side of a paw is exposed to a luminous source, generating a temperature of, e. g. 55° C. Thermal sensitivity is measured as latency between start of exposure and lifting/pulling the exposed paw.

Mice treated with a CDK9 inhibitor as disclosed herein and carrageenan, or with Naproxen and carrageenan, or with solvent and carrageenan, respectively, are subjected to a Hargreaves assay. Mice treated with a CDK inhibitor and carrageenan could display a longer latency, compared to negative control mice. This observation would be indicative for a hypoalgesic effect of the CDK inhibitors as disclosed herein.

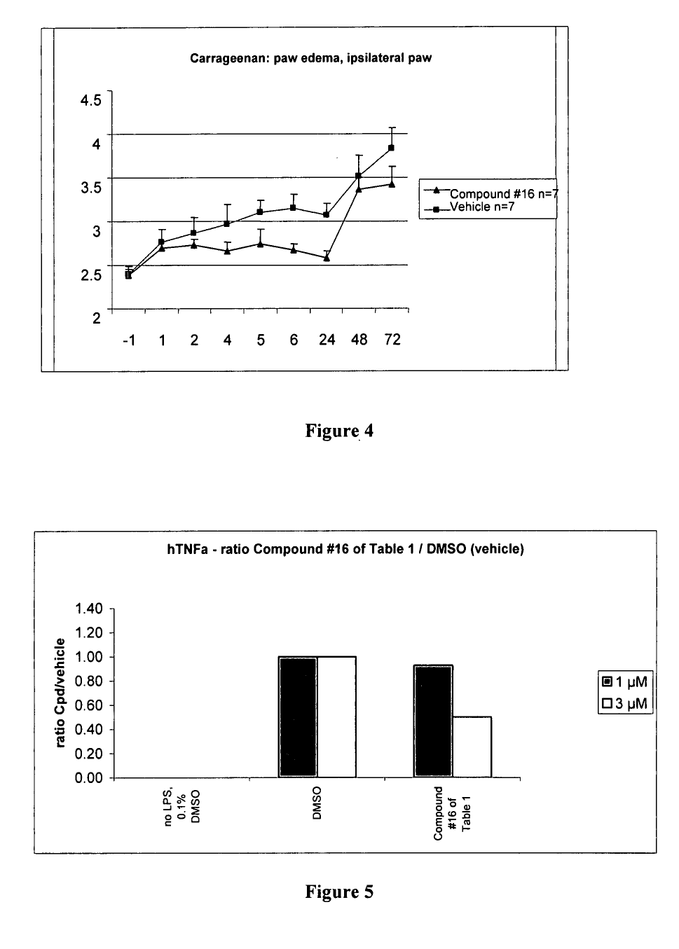

Compound #16 of Table 1 was used in the carrageenan assay as described above and the effect on paw swelling was investigated. The results of administration of compound #16 of Table 1 are shown in FIG. 4.

FIG. 4 depicts the results of ipsi-lateral paw edema measurements performed with carrageenan-treated mice. Within the observation period all animals treated with compound #16 of Table 1 showed reduced paw edema compared to the vehicle control group.

### Example 5

### Carrageenan Assay in Rats—Model of Inflammation and Inflammatory Pain

The following depicts one possible way of performing the carrageenan assay in rats.

Said assay detects analgesic/anti-inflammatory activity in rats with inflammatory pain, following the protocol as described by Winter et al (Proc. Soc. Exp. Biol. Med., 111, 544-547, 1962).

Rats (200-250 g) are injected with a suspension of carrageenan into the lower surface of the right hindpaw (0.75 mg per paw in 0.05 ml physiological saline). Two hours later rats are submitted consecutively to tactile and thermal stimulation of both hindpaws.

For tactile stimulation, the animal is placed under an inverted acrylic plastic box (18×11.5×13 cm) on a grid floor. The tip of an electronic Von Frey probe (Bioseb, Model 1610) is then applied with increasing force first to the non-inflamed and then the inflamed hindpaw and the force required to induce paw-withdrawal is automatically recorded. This procedure is carried out 3 times and the mean force per paw is calculated.

For thermal stimulation, the apparatus (Ugo Basile, Reference: 7371) consists of individual acrylic plastic boxes (17×11×13 cm) placed upon an elevated glass floor. A rat is placed in the box and left free to habituate for 10 minutes. A mobile infrared radiant source (96±10 mW/cm²) is then focused first under the non-inflamed and then the inflamed hindpaw and the paw-withdrawal latency is automatically recorded. In order to prevent tissue damage the heat source is automatically turned off after 45 seconds.

After the behavioral measures, the paw edema is evaluated by measuring the volume of each hindpaw using a digital plethysmometer (Letica, Model 7500), which indicates water displacement (in ml) induced by paw immersion.

10 rats are studied per group. The test is performed blind.

The test substance, such as a CDK inhibitor according to any one of general Formula I, II or III as presented herein, will be evaluated at 2 doses (10 and 30 mg/kg), administered p.o. 60 minutes before the test, and compared with a vehicle control group.

Morphine (128 mg/kg p.o.) and acetylsalicylic acid (512 mg/kg p.o.), administered under the same experimental conditions, will be used as reference substances.

The experiment will therefore include 6 groups. Data will be analyzed by comparing treated groups with vehicle control using unpaired Student's t tests.

Rats treated with a CDK9 inhibitor as disclosed herein and carrageenan, or with Naproxen and carrageenan, or with solvent and carrageenan, respectively, are subjected to a Hargreaves assay. Rats treated with a CDK inhibitor and carrageenan should display a longer latency, compared to negative control rats. This observation would be indicative for a hypoalgesic effect of the CDK inhibitors as disclosed herein.

### Example 6

### LPS In Vivo Assay (LPS)—Model of Cytokine Repression In Vivo

For the LPS induced model of septic shock, mice receive an intraperitoneal (i.p.) injection of 30 μg bacterial Lipopolysaccharide (LPS; L2630 SIGMA) in saline. Said LPS-mediated initiation of the inflammatory signalling cascade results in increasing blood serum concentrations of cytokines such as e.g. TNFα, IL-6 and IL1β. Blood can be taken from these animals at defined time points. Thereafter, serum will be separated and the samples can be stored at −80° C. until cytokine concentrations are measured using commercial ELISA assays. (Al Moreira et al., Braz J Med Biol Res 1997; 30:1199-1207).

It has been recognized that inflammatory mediators such as the cytokines TNFα, IL6 and IL1β can contribute to persistent pain states as well as inflammatory disorders. After being released from immune cells like macrophages in peripheral and microglia in CNS tissues, these mediators seem to play a pivotal role not only in inflammatory and neuropathic pain but also in inflammatory disorders such as rheumatoid arthritis (F Marchand et al., Nat Rev Neurosci 2005; 6 (7); 521-532). Thus, inhibition of tumor necrosis factor α (TNFα) represents a relevant target for the treatment of inflammatory diseases as well [Lavagno et al., Eur J Pharmacol 2004; 501, 199-208].

The LPS in vivo assay can be used as a powerful model to address repression of cytokine expression by pharmacological treatments.

### 1. Induction of Cytokine Expression in Wildtype Mice

Wildtype mice (strain C3HeB/FeJ) (age, sex and weight matched) are injected with 30 μg LPS (SIGMA) intraperitoneally. 90 minutes after LPS administration these animals are anaesthetized with 0.1 ml/10 g bodyweight Ketamine-Rompun (50/20 mg/ml) and blood for serum preparation is taken via cardiac puncture.

### 2. Administration of CDK-Inhibiting Compounds to LPS Mice

Pharmacological treatment groups (n=4) of LPS mice receive per os (p.o.) injections of CDK-inhibiting compounds or the vehicle (negative control), respectively.

10 or 30 mg/kg (compound per bodyweight) of a CDK inhibitor, dissolved in 1% carboxy-Methylcellulose (SIGMA) is administered as a single p.o. dosage 30 min prior to LPS stimulation. Vehicle control is administered in the same manner.

90 minutes (min) after LPS stimulation, blood samples are taken from the mice. Previously, the 90 min time point had been identified as the peak of TNF alpha expression in this animal model by a time course experiment.

The effect of pharmacological treatment with CDK inhibitors on cytokine levels in LPS mice is analyzed in commercial ELISA assays as described below.

### 3. Determination of Cytokine Blood Serum Concentrations in LPS Mice After Administration of CDK-Inhibiting Compounds

Blood samples (˜500 μl/animal) from the LPS animals are incubated on wet ice for 30 min after cardiac puncture. Afterwards the samples are centrifuged for 15 min at 13,000 rpm. Serum is separated from the clot and stored frozen at −80° C.

Serum concentrations of TNF alpha and IL6 within the samples are measured by using commercial ELISA Kits (Natutec) according to the manufacturers instructions.

### 4. Effects of Administration of CDK-Inhibiting Compounds on the Protein Expression of Cytokines

Compounds according to any one of general Formula I, II or III are administered to LPS mice as described above. ELISA based determinations of cytokine serum concentrations are performed as described above. Comparison of animals treated with compounds of the present invention versus vehicle treated control animals should display a significant repressive effect on TNFα and IL6 protein concentration in the blood serum. Said finding would indicate that a compound according to the present invention is effective as a suppressive drug of cytokines TNF alpha and IL6 in models of cytokine expression.

### Example 7

### In Vitro THP-1 Assay—In Vitro Model of Cytokine Inhibition

The human THP-1 cell line can be utilized as an in vitro model of cytokine expression as mediated by Lipopolysaccharide (LPS) or Tumor Necrosis Factors [TNFα].

Monocytic THP-1 cells (ATCC; TIB-202) can be differentiated into macrophage-like cells expressing pro-inflammatory cytokines like TNFα, IL6 and IL1β upon induction with LPS or by TNFα (autocrine induction) itself.

It has been recognized that inflammatory mediators such as the cytokines TNFα, IL6 and IL1β can contribute to persistent pain states as well as to inflammatory disorders. After being released from immune cells like macrophages in peripheral and microglia in CNS tissues, these mediators seem to play a pivotal role not only in inflammatory and neuropathic pain but also in inflammatory disorders such as rheumatoid arthritis (F Marchand et al., Nat Rev Neurosci 2005; 6 (7); 521-532). Hence inhibition of tumor necrosis factor α (TNFα) represents a relevant target in the treatment of inflammatory disorders as well [Lavagno et al., Eur J Pharmacol 2004; 501, 199-208].

Therefore, the THP-1 in vitro assay can be used as a powerful screening model to address pharmacological inhibition of cytokine expression (U Singh et al, Clin Chem 2005; 51 (12); 2252-6], K Rutault et al., J Biol Chem 2001; 276 (9); 6666-74].

### 1. Growth and Differentiation of THP-1 Cells

THP-1 cells are grown in modified RPMI-1640medium (ATCC, Cat. No. 30-2001) supplemented with 10% FCS and 1% Pen/Strep. For cytokine inhibition assays, cells are seeded at a density of 5×10⁵ cells/ml into 6-well plates in standard growth medium supplemented with 100 ng/ml PMA (Sigma, P1585) to induce differentiation into macrophage-like cells. After 24 hours, the medium is replaced with standard growth medium (without PMA) and the cells are incubated for another 48 hours to complete differentiation.

### 2. Treatment of Differentiated THP-1 Cells With CDK-Inhibiting Compounds and LPS Stimulation

After 72 h of differentiation, the medium is replaced with serum free growth medium, and CDK-inhibiting compounds as well as reference compounds such as positive and negative controls, each dissolved in DMSO are added at concentrations ranging from 0.5 to 5 μM (final concentration of DMSO in the well is 0.1%). Cells are incubated for 60 min with compounds prior to stimulation with 100 ng/ml LPS (Sigma, L2630) for another 4-48 hours. Supernatants are collected and assayed immediately for cytokine expression, e.g. for TNFα, IL-6 and IL-1b using commercially available sandwich ELISA assays (eBioscience, Cat. No 88-7346, 88-7066, 88-7010) or kept frozen at 20° C. until evaluation.

### 3. Determination of Cytokine Concentrations in THP-1 Supernatant After Administration of CDK-Inhibiting Compounds

Concentrations of TNFα, IL6 and IL1β within the cell culture supernatants are measured by using commercial ELISA Kits (eBioscience) according to the manufacturer's instructions.

### 4. Effects of Treatment With CDK-Inhibiting Compounds on the Protein Expression of Cytokines in THP-1 Cell Supernatants

CDK-inhibitory compounds according to any one of general Formula I, II or III are administered to differentiated THP-1 cells in triplicates as described above (see section 2.). After 60 min of pre-incubation with test or reference compound (SB203580, a p38 inhibitor and BMS345541, an IKK-inhibitor) alone, cells are stimulated with LPS. After incubation for 4-48 h, supernatants are collected and ELISA based determinations of cytokine supernatant concentrations are performed as described in section 3, supra.

Comparison of cells treated with compounds according to any one of general Formula I, II or III and reference compounds versus cells treated with vehicle (DMSO) should display a significant inhibitory effect of the CDK-inhibitory compounds on TNFα and IL6 protein concentration in the cell supernatant. Compared to reference compounds SB203580 or BMS3455541, these compounds should exhibit a similar or better inhibition of TNFα/Il-6 expression.

Specifically, compound #16 of Table 1 was administered to differentiated THP-1 cells in triplicates as described above. After 60 min of pre-incubation with test or reference compound (a p38 inhibitor and an IKK-inhibitor, respectively) alone, cells were stimulated with LPS. After incubation for 4-48 h, supernatants were collected and ELISA based determinations of cytokine supernatant concentrations were performed as described in section 3, supra.

Comparison of cells treated with compound #16 of Table 1 versus cells treated with vehicle (DMSO) displayed a significant inhibitory effect of compound #16 of Table 1 on TNFα and IL6 protein concentration in the cell supernatant. Compared to the reference compounds, compound #16 of Table 1 exhibited a similar or better inhibition of TNFα/Il-6 expression.

The effects of administration of compound #16 of Table 1 on expression of TNFα in LPS induced THP-1 macrophages are shown in FIG. 5.

These findings indicate that CDK-inhibitory compound #16 of Table 1 is an effective suppressor of expression of cytokines TNFα and IL-6.

FIG. 5 shows reduction of LPS-induced cytokine levels by compound #16 of Table 1, exemplified for TNFα levels. At a concentration of 3 μM, compound #16 of Table 1 reduces the TNFα signal to 50% compared to the DMSO (vehicle control). These findings indicate that CDK-inhibitory compound #16 of Table 1 is an effective suppressor of expression of LPS-induced cytokines.

### Example 8

### In Vitro Human Whole Blood Assay—In Vitro Model of Cytokine Inhibition

The aim of this in vitro assay is to profile anti-inflammatory compounds for their ability to inhibit Lipopolysaccharide (LPS) or Staphylococcus Enterotoxin B (SEB) induced expression of cytokines (e.g. TNFα, IL-1β, IFNγ, IL-2, IL-4, IL-6, IL-8, IL-10, IL-13) in human whole blood. For this purpose, human heparinised whole blood from healthy donors was diluted 1:5 with medium (RPMI 1640, e.g. ATCC #30-2001, supplemented with 4.5 g/L glucose, 4 mM L-Glutamine, pyruvate, 1250 U Sodium-Heparin and 1% Penicillin/Streptomycin), incubated with the anti-inflammatory compound or solvent for 30-60 minutes and then stimulated with 20 ng/ml to 1 μg/ml LPS or SEB for 3 to 48 hours. Supernatants were harvested and analysed for cytokine content, e.g. by ELISA.

### Results

In the human whole blood assay, TNF α levels were reduced by compound #16 of Table 1 by 89% at a concentration of 10 μM, 85% at a concentration of 5 μM, and 78% at a concentration of 0.5 μM. From this data an IC50 value below 0.5 μM can be deduced. These findings indicate that CDK-inhibitory compound #16 of Table 1 is an effective suppressor of expression of Lipopolysaccharide (LPS) or Staphylococcus Enterotoxin B (SEB) induced cytokines.

### Example 9

### In Vitro Kinase Inhibition Assays

IC50 profiles of the compounds according to general Formula I, II or III of the present invention can be determined for cyclin-dependent kinases CDK2/CycA, CDK4/CycD1, CDK6/CycD1 and CDK9/CycT in enzymatic kinase inhibition assays in vitro. IC50 values as obtained in these assays are used for evaluating the specific selectivity and potency of the compounds with respect to CDK9 inhibition.

Results obtained in these assays are used to select compounds displaying specificity for CDK9. Specifically, it is intended to distinguish the CDK9-specific compounds from other compounds having significant inhibitory potency also with regard to other CDKs, i.e. on some or all of CDKs 2, 4 and 6. This separation is essential in order to avoid adverse (cytostatic/cytotoxic) effects, which may occur upon inhibition of cell cycle relevant CDKs 2, 4 and 6.

Furthermore, these data are used to establish structure activity relationships (SAR) supporting the design of new and even improved structures/compounds with respect to potency and selectivity.

Specifically, the IC50 profile of compound #16 of Table 1 was determined for cyclin-dependent kinases CDK9/CycT in enzymatic kinase inhibition assays in vitro.

### 1. Test Compounds

Compounds are used as 1×10⁻⁰² M stock solutions in 100% DMSO, 100 μl each in column 2 of three 96-well V-shaped microtiterplates (in the following, said plates are referred to as “master plates”).

Subsequently, the 1×10⁻⁰² M stock solutions in column 2 of the master plates are subjected to a serial, semi-logarithmic dilution using 100% DMSO as a solvent, resulting in 10 different concentrations, the dilution endpoint being 3×10⁻⁰⁷ M/100% DMSO in column 12. Column 1 and 7 are filled with 100% DMSO as controls. Subsequently, 2×5 μl of each well of the serial diluted copy plates are aliquoted in 2 identical sets of “compound dilution plates”, using a 96-channel pipettor.

On the day of the kinase inhibition assay, 45 μl H₂O are added to each well of a set of compound dilution plates. To minimize precipitation, H₂O is added to the plates only a few minutes before the transfer of the compound solutions into the assay plates. The plates are shaken thoroughly, resulting in “compound dilution plates/10% DMSO” with a concentration of 1×10⁻⁰³ M/10% DMSO to 3×10⁻⁰⁸ M/10% DMSO in semilog steps. These plates are used for the transfer of 5 μl compound solution into the “assay plates”. The compound dilution plates are discarded at the end of the working day. For the assays (see below), 5 μl solution from each well of the compound dilution plates are transferred into the assay plates. The final volume of the assay is 50 μl. All compounds are tested at 10 final assay concentrations in the range from 1×10⁻⁰⁴ M to 3×10⁻⁰⁹ M. The final DMSO concentration in the reaction mixtures is 1% in all cases.

### 2. Recombinant Protein Kinases

For the determination of inhibitory profiles, the following 4 protein kinases are used: CDK2/CycA, CDK4/CycD1, CDK6/CycD1 and CDK9/CycT. Said protein kinases are expressed in Sf9 insect cells as human recombinant GST-fusion proteins or His-tagged proteins by means of the baculovirus expression system. Kinases are purified by affinity chromatography using either GSH-agarose (Sigma) or Ni—NTH-agarose (Qiagen). The purity of each kinase is determined by SDS-PAGE/silver staining and the identity of each kinase is verified by western blot analysis with kinase specific antibodies or by mass spectroscopy.

### 3. Protein Kinase Assay

All kinase assays are performed in 96-well FlashPlates™ from Perkin Elmer/NEN (Boston, Mass, USA) in a 50 μl reaction volume. The reaction mixture is pipetted in four steps in the following order:

1. 20 μl of assay buffer (standard buffer)

2. 5 μl of ATP solution (in H₂O)

3. 5 μl of test compound (in 10% DMSO)

4. 10 μl of substrate/10 μl of enzyme solution (premixed)

The assay for all enzymes contains 60 mM HEPES-NaOH, pH 7.5, 3 mM MgCl₂, 3 mM MnCl₂, 3 μm Na-Orthovanadate, 1.2 mM DTT, 50 μg/ml PEG20000, 1 μM [γ-³³P]-ATP (approx. 5×10⁰⁵ cpm per well).

The following amounts of enzyme and substrate are used per well:

| | | Kinase | Kinase | | Substrate |
|---|---|---|---|---|---|
| # | Kinase | Lot # | ng/50 μl | Substrate | ng/50 μl |
| 1 | CDK2/CycA | P005 | 100 | Histone H1 | 250 |
| 2 | CDK4/CycD1 | SP005 | 50 | Rb-CTF (Lot 009) | 500 |
| 3 | CDK6/CycD1 | SP003 | 400 | Rb-CTF (Lot 009) | 500 |
| 4 | CDK9/CycT | SP003 | 100 | Rb-CTF (Lot 009) | 1000 |

Reaction mixtures are incubated at 30° C. for 80 minutes. The reaction is stopped with 50 μl of 2% (v/v) H₃PO₄, plates are aspirated and washed two times with 200 μl H₂O or 200 μl 0.9% (w/v) NaCl. Incorporation of ³³P is determined with a microplate scintillation counter (Microbeta, Wallac).

All assays are performed with a BeckmanCoulter/Sagian robotic system.

### 4. Evaluation of Raw Data

The median value of the counts in column 1 (n=8) of each assay plate is defined as “low control”. This value reflects unspecific binding of radioactivity to the plate in the absence of a protein kinase but in the presence of the substrate. The median value of the counts in column 7 of each assay plate (n=8) is taken as the “high control”, i.e. full activity in the absence of any inhibitor. The difference between high and low control ss referred to as 100% activity. As part of the data evaluation, the low control value from a particular plate was subtracted from the high control value as well as from all 80 “compound values” of the corresponding plate. The residual activity (in %) for each well of a particular plate is calculated by using the following Formula:

Res. Activity (%)=100×[(cpm of compound−low control)/(high control−low control)]

The residual activities for each concentration and the compound IC50 values are calculated using Quattro Workflow V2.0.1.3 (Quattro Research GmbH, Munich, Germany; www.quattro-research.com). The model used is “Sigmoidal response (variable slope)” with parameters “top” fixed at 100% and “bottom” at 0%.

### 5. Results

The IC50 value on CDK9/CycT for compound #16 of Table 1 was 51 nM.

### Selected Preferred Embodiments

The following are selected preferred embodiments of the methods and compositions of the invention.

A. A method for the treatment of pain comprising administering to a subject in need thereof an amount of at least one inhibitor of a cyclin-dependent kinase (CDK) sufficient to ameliorate pain in said subject, wherein the at least one inhibitor of a cyclin-dependent kinase comprises a thiazole of general Formula I, II or III:

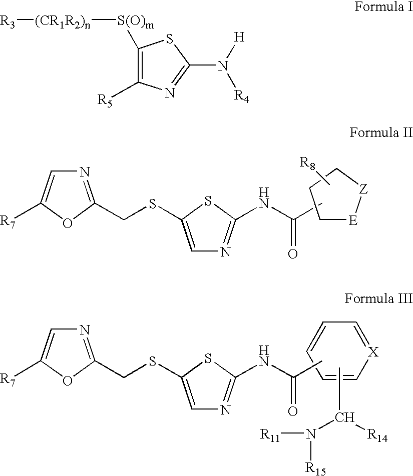

as described herein.

B. The method according to A, wherein the thiazole is characterized by general Formula I, wherein

R₁ and R₂ are independently hydrogen, fluorine or alkyl;

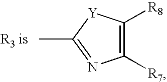

wherein Y is oxygen, sulfur or NR₉;

R₄ is alkyl, cycloalkyl, aryl, cyoloalkylalkyl, arylalkyl, heteroaryl, heteroarylalkyl, heterocycloalkyl, heterocycloalkylalkyl, or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkyl-heteroaryl, C(NNO₂)NH-heterocyloalkyl, C(NNO₂)NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHCO alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₆)NH-alkyl, C(NOR₆)NH-cycloalkyl, C(NOR₆)NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;

R₅ is hydrogen or alkyl;

R₆ is hydrogen, alkyl, cycloalkyl, aryl, cycloalkylalkyl, arylalkyl, heteroaryl, heteroarylalkyl, heterocycloalkyl or heterocycloalkylalkyl;

R₇ and R₈ are independently hydrogen, alkyl, substituted alkyl, cycloalkyl, aryl, substituted aryl, cycloalkylalkyl, arylalkyl, heteroaryl, substituted heteroaryl, heteroarylalkyl, heterocycloalkyl, heterocycloalkyalkyl; and

R₉ is hydrogen, alkyl, cycloalkyl, aryl, alkylcycloalkyl, arylalkyl, heteroaryl, heteroarylalkyl, heterocycloalkyl or heterocycloalkylalkyl.

C. The method according to A, wherein the thiazole is characterized by general Formula I, wherein

R₁ and R₂ are independently hydrogen, fluorine or alkyl;

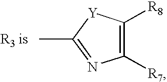

wherein Y is oxygen;

R₄ is alkyl, cycloalkyl, aryl, cycloalkylalkyl, arylalkyl, heteroaryl, heteroarylalkyl, heterocycloalkyl, heterocycloalkylalkyl; or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-cycloalkyl, SO₃-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloaIkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkyl-heteroaryl, C(NNO₂)NH-heterocyloalkyl, C(NNO₂)NH-alkyl-heterocycloalkyl; or C(NH)NH-alkyl, C(NH)NH-cycloalkyl, C(NH)NH-ayrl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NNOH)NHCO-aryl, C(NH)NHCO-alkyl-cycloalkyl, C(NH)NHCO alkyl-aryl; C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₆)NH-alkyl, C(NOR₂)NH-cycloalkyl, C(NOR₆)NH-aryl, C(NOR₅)NH-alkyl-cycloalkyl, C(NOR₆)NH-alkyl-aryl, C(NOR₅)NH-heteroaryl, C(NOR₅)NH-alkyl-heteroaryl, C(NOR₆)NH-heterocylcoalkyl, C(NOR₅)NH-alkyl-heterocycloalkyl;

R₅ is hydrogen;

R₆ is hydrogen, alkyl, cycloalkyl, aryl, cyclolkylalkyl, arylalkyl, heteroaryl, heteroarylalkyl, heterocycloalkyl or heterocycloalkylalkyl; and

R₇ and R₈ are independently hydrogen, alkyl, substituted alkyl, cycloalkyl, aryl, subsituted aryl, cycloal kylalkyl, arylalkyl, heteroaryl, substituted heteroaryl, heteroarylalkyl, heterocycloalkyl, heterocycloalkyalkyl.

D. The method according to A, wherein the thiazole is characterized by general Formula I, wherein

R₁ and R₂ are independently hydrogen, fluorine or alkyl;

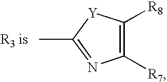

wherein Y is sulfur;

R₄ is alkyl, cycloalkyl, aryl, cycloalkylalkyl, arylalkyl, heteroaryl, heteroarylalkyl, heterocycloalkyl, heterocycloalkylalkyl, or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl: or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-cycloalkyl, SO₂-aryl, SO₂-alkyl-cyloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)OH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkyl-heteroaryl, C(NNO₂)NH-heterocyloalkyl, C(NNO)NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHCO-alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₆)NH-alkyl, C(NOR₆)NH-cycloalkyl, C(NOR₆)NH-aryl, C(NOR₆)NH-alkylcycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;

R₅ is hydrogen;

R₆ is hydrogen, alkyl, cycloalkyl, aryl, cycloalkylalkyl, arylalkyl, heteroaryl, heteroarylalkyl, heterocycloalkyl or heterocycloalkylalkyl; and

R₇ and R₈ are independently hydrogen, alkyl, substituted alkyl, cycloalkyl, aryl, substituted aryl, cycloalkylalkyl, arylalkyl, heteroaryl, substituted heteroaryl, heteroarylalkyl, heterocycloalkyl, heterocycloalkyalkyl.

E. The method according to A, wherein the thiazole is characterized by general Formula I, wherein

R₁ and R₂ are independently hydrogen, fluorine or alkyl;

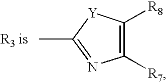

wherein Y is NR₉;

R₄ is alkyl, cycloalkyl, aryl, cycloalkylalkyl, arylalkyl, heteroaryl, heteroarylalkyl, heterocycloalkyl, heterocycloalkylalkyl; or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkylheteroaryl, C(NNO₂)NH-heterocyloalkyl, C(NNO₂)NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHCO-alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₈)NH-alkyl, C(NOR₆)NH-cycloalkyl, C(NOR₆)NH-aryl, C(NOR₆)NH-alkyl-cycloalkyl, C(NOR₅)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;

R₅ is hydrogen,

R₆ is hydrogen, alkyl, cycloalkyl, aryl, cycloalkylalkyl, arylalkyl, heteroaryl, heteroarylalkyl, heterocycloalkyl or heterocycloalkylalkyl;

R₇ and R₈ are independently hydrogen, alkyl, substituted alkyl, cycloalkyl, aryl, subsituted aryl, cycloalkylalkyl, arylalkyl, heteroaryl, substituted heteroaryl, heteroarylalkyl, heterocycloalkyl, heterocycloalkylalkyl; and

R₉ is hydrogen, alkyl, cycloalkyl, aryl, cycloalkylalkyl, arylalkyl, heteroaryl, heteroarylalkyl, heterocycloalkyl or heterocycloalkylalkyl.

F. The method according to A, wherein the thiazole is characterized by general Formula I, wherein

R₁ and R₂ are independently hydrogen, fluorine or alkyl;

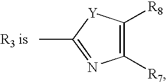

wherein Y is oxygen;

R₄ is CO-alkyl, CO-alkyl-aryl, CO-cycloalkyl, CO-alkyl-heteroaryl, CO-alkyl-heteroalkyl, CO-alkyl-heterocycloalkyl, CONH-alkyl, CONH-alkyl-aryl, CONH-cycloalkyl, or CONH-alkyl-heterocycloalkyl;

R₅ is hydrogen; and

R₇ and R₈ are hydrogen;

m is 0; and

n is 1.

G. The method according to A, wherein the thiazole is characterized by general Formula I, wherein

R₁ and R₂ are independently hydrogen, fluorine or alkyl;

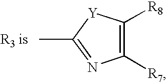

wherein Y is oxygen;

R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;

R₅ is hydrogen;

R₇ and R₈ are alkyl;

m is 0; and

n is 1.

H. The method according to A, wherein said at least one inhibitor of a cyclin-dependent kinase is characterized by general Formula I, wherein

R₁ and R₂ are independently hydrogen, fluorine or alkyl;

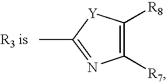

wherein Y is oxygen;

R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;

R₅ is hydrogen;

R₇ is hydrogen;

R₈ is alkyl;

m is 0; and

n is 1.

I. The method according to A, wherein the thiazole is characterized by general Formula I, wherein

R₁ and R₂ are independently hydrogen, fluorine or alkyl;

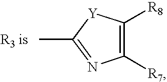

wherein Y is oxygen;

R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;

R₅ is hydrogen;

R₇ is alkyl;

R₈ is hydrogen;

m is 0; and

n is 1.

J. The method according to A, wherein the thiazole is characterized by general Formula I, wherein

R₁ and R₂ are independently hydrogen, fluorine or alkyl;

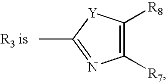

wherein Y is sulfur;

R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;

R₅ is hydrogen;

R₇ is hydrogen;

R₈ is alkyl;

m is 0; and

n is 1.

K. The method according to A, wherein the thiazole is characterized by general Formula I, wherein

R₁ and R₂ are independently hydrogen, fluorine or alkyl;

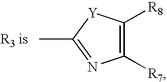

wherein Y is sulfur;

R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;

R₅ is hydrogen;

R₇ is alkyl;

R₈ is hydrogen;

m is 0; and

n is 1.

L. The method according to A, wherein the thiazole is characterized by general Formula I, wherein

R₁ and R₂ are independently hydrogen, fluorine or alkyl;

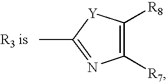

wherein Y is NR₉;

R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;

R₅ is hydrogen;

R₇ is hydrogen;

R₈ is alkyl;

R₉ is hydrogen, alkyl, cycloalkyl, aryl, alkyl-cycloalkyl, alkyl-aryl, heteroaryl, alkyl-heteroaryl, heterocycloalkyl, or alkyl-heterocycloalkyl;

m is 0; and

n is 1.

M. The method according to A, wherein the thiazole is characterized by general Formula I, wherein

R₁ and R₂ are independently hydrogen, fluorine or alkyl;

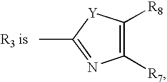

wherein Y is NR₉;

R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;

R₅ is hydrogen;

R₇ is alkyl;

R₈ is hydrogen;

R₉ is alkyl;

m is 0; and

n is 1.

N. The method according to A, wherein the thiazole is characterized by general Formula I, wherein

R₁ and R₂ are independently hydrogen, fluorine or alkyl;

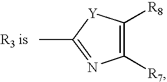

wherein Y is NR₉;

R₄ is CO-alkyl, CO-alkyl-aryl, CO-cycloalkyl, CO-alkyl-heteroaryl, CO-alkyl-heteroalkyl, CO-alkyl-heterocycloalkyl, CONH-alkyl, CONH-alkyl-aryl, CONH-cycloalkyl, or CONH-alkyl-heterocycloalkyl;

R₅ is hydrogen;

R₇ is alkyl;

R₈ is hydrogen;

R₉ is hydrogen;

m is 0

n is 1.

O. The method according to A, wherein the thiazole is characterized by general Formula I, wherein said inhibitor is selected from the group consisting of:
N-[5-[[5-Ethyl-2-oxazolyl)methyl]thio]-2-thiazoly]acetamide;
N-[5-[[5-Ethyl-2-oxazolyl)methyl]thio]-2-thiazolyl]benzamide;
N-[5-[[5-Ethyl-2-oxazolyl)methyl]thio]-2-thiazolyl]benzenesulfonamide;
N-[5-[[(4,5-Dimethyl-2-oxazolyl)methyl]thio]-2-thiazolyl]acetamide;
N-[5-[[(5-t-Butyl-2-oxazolyl)methyl]thio]-2-thiazolyl]acetamide;
N-[5-[[5-t-Butyl-2-oxazolyl)methyl]thio]-2-thiazolyl]trimethylacetamide;
N-[5-[[(4-Ethyl-2-oxazolyl)methyl]thio]-2-thiazolyl]acetamide;
N-[5-[[(5-t-Butyl-2-oxazolyl)methyl]thio]-2-thiazolyl]-N′-cyano-N″-(2,6-difluorophenyl)guanidine;
N-[5-[[(5-isopropyl-2-oxazolyl)fluoromethyl]thio]-2-thiazolyl]acetamide;
N-[5-[[(5-t-Butyl-2-oxazolyl)methyljthio]-2-thiazolyl]aminophenyl-4-(2-hydroxyethyl)sulfonamide;
N-[5-[[(5-t-Butyl-2-oxazolyl)methyl]thio]-2-thiazolyl]aminophenyl-4-sulfonamide;
N-[5-[[(5-t-Butyl-2-oxazolyl)methyl]thio]-2-thiazolyl]-4-aminopyrimidine;
N-[5-[[(5-t-Butyl-2-oxazolyl)methyl]thio]-2-thiazolyi]-3-(hydroxymethyl)aniline;
N-[5-[[(5-t-Butyl-2-oxazolyl)methyl]thio]-2-thiazolyl]-2-aminopyridine;
N-[5-[[(5-t-Butyl-2-oxazolyl)methyl]thio]-2-thiazolyl]-2-[5-[(((3-hydroxy-2,2-dimethyl)propyl)amino)methyl]]aminopyridine;
N-[5-[[[5-(1,1-dimethylethyl)-2-oxazolyl]-methyl]thio]-2-thiazolyl]-4-piperidinecarboxamide, or a pharmaceutically acceptable salt thereof.

P. The method according to A, wherein the thiazole is characterized by general Formula II:

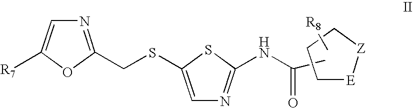

wherein R₇ is alkyl; R₈ is hydrogen or alkyl; Z is NR₉ or CHNR₉R₁₀; R₉ and R₁₀ are each independently hydrogen, alkyl, substituted alkyl, cycloalkyl or substituted cycloalkyl; E is a covalent bond or (CH₂)ₚ; and p is 1, 2 or 3; and enantiomers, diastereomers and pharmaceutically acceptable salts thereof.

Q. The method according to P, wherein the thiazole is characterized by general Formula II, wherein R₇ is alkyl; R₈ is hydrogen; Z is NR₉ or CHNR₉R₁₀; R₉ and R₁₀ are each independently hydrogen, alkyl, substituted alkyl or cycloalkyl; E is (CH₂)ₚ, and p is 2.

R. The method according to P, wherein the thiazole is characterized by general Formula IIa:

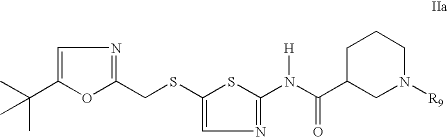

wherein R₉ is hydrogen, alkyl, substituted alkyl or cycloalkyl and enantiomers, diastereomers and pharmaceutically acceptable salts thereof.

S. The method according to P, wherein the thiazole is characterized by general Formula IIb:

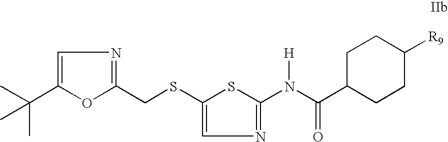

wherein R₉ is alkyl, substituted alkyl or cycloalkyl and enantiomers, diastereomers and pharmaceutically acceptable salts thereof.

T. The method according to claim P, wherein the thiazole is characterized by general Formula IIc:

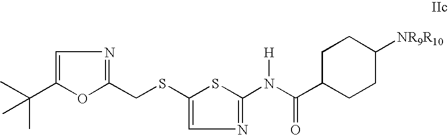

wherein R₉ and R₁₀ are each independently hydrogen, alkyl, substituted alkyl or cycloalkyl with the proviso that both R₉ and R₁₀ cannot be hydrogen and enantiomers, diasteromers and pharmaceutically acceptable salts thereof.

U. The method according to P, wherein the thiazole is characterized by general Formula II, wherein said inhibitor is selected from the group consisting of:
(±)-N-[5-[[[5-(1,1-dimethylethyl)-2-oxazolyl]methyl]thio]-2-thiazolyl]-3-piperidinecarboxamide;
(±)-1-(2,3-dihydroxypropyl)-N-[5-[[[5-(1,1-dimethylethyl)-2-oxazolyl]methyl]thio]-2-thiazolyl]-4-piperidinecarboxamide;
N-[5-[[[5-(1,1-dimethylethyl)-2-oxazolyl]methyl]thio]-2-thiazolyl]-1-(1-methylethyl)-4-piperidinecarboxamide;
1-cyclopropyl-N-[5-[[[5-(1,1-dimethylethyl)-2-oxazolyl]methyl]thio]-2-thiazolyl]-4-piperidinecartoxamide;
N-[5-[[[5-(1,1-dimethylethyl)-2-oxazolyl]methyl]thio]-2-thiazolyl]-1-(2-hydroxyethyl)-4-piperidinecarboxamide;
(R)—N-[5-[[[5-(1,1-dimethylethyl)-2-oxazolyl]methyl]thio]-2-thiazolyl]-3-piperidinecarboxamide;
(S)—N-[5-[[[5-(1,1-dimethylethyl)-2-oxazolyl]methyl]thio]-2-thiazolyl]-3-piperidinecarboxamide;
cis-4-amino-N-[5-[[[5-(1,1-dimethylethyl)-2-oxazolyl]methyl]thio]-2-thiazolyl]cyclohexylcarboxamide; and
trans-4-amino-N-[5-[[[5-(1,1-dimethylethyl)-2-oxazolyl]methyl]thio]-2-thiazolyl]cyclohexylcarboxamide; and
   pharmaceutically acceptable salts thereof.

V. The method according to claim A, wherein the thiazole is characterized by general Formula III:

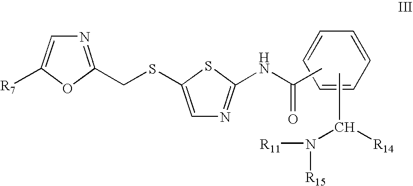

wherein

R₇ is alkyl;

R₈ and R₁₄ are each independently hydrogen or alkyl;

R₁₅ is hydrogen or alkyl, and R₁₁ is hydrogen or alkyl substituted with one or two hydroxy groups or one NR₁₂R₁₃ group; or R₁₅ and R₁₁ are taken together with the nitrogen atom to which they are attached to form a 4- to 7-membered heterocyclic ring wherein R₁₅R₁₁ is represented by —(CH₂)_{q}— where q is an integer of 3, 4, 5 or 6;

R₁₂ and R₁₃ are each independently hydrogen, alkyl, substituted alkyl, cycloalkyl or substituted cycloalkyl, or R₁₂ and R₁₃ are taken together with the nitrogen atom to which they are attached to form a 4- to 7-membered heterocyclic ring wherein R₁₂R₁₁ is represented by —(CH₂)_{q}— where q is an integer of 3, 4, 5 or 6; and

X is CH or N,

and enantiomers, diastereomers and pharmaceutically acceptable salts thereof.

W. The method according to V, wherein the thiazole is characterized by general Formula III, wherein

R₇ is tert-butyl;

R₈ and R₁₄ are each independently hydrogen or methyl;

R₁₅ is hydrogen, and R₁₁ is hydrogen, —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

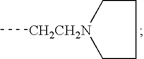

or R₁₅ and R₁₁ are taken together with the nitrogen atom to which they are attached to form a 5-membered heterocyclic ring where R₁₅R₁₁ is represented by —(CH₂)₄—; and

X is CH or N.

X. The method according to V, wherein the thiazole is characterized by general Formula IIIa:

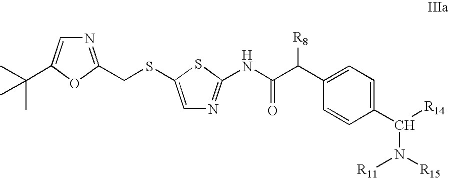

wherein

R₈ and R₁₄ are each independently hydrogen or methyl; and

R₁₅ is hydrogen, and R₁₁ is hydrogen, —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

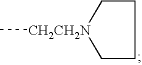

or

R₁₅ and R₁₁ are taken together with the nitrogen atom to which they are attached to form a 5-membered heterocyclic ring where R₁₅R₁₁ is represented by —CH₂)₄—, and enantiomers, diastereomers and pharmaceutically acceptable salts thereof.

Y. The method according to X, wherein

R₈ and R₁₄ are each independently hydrogen or methyl; and

R₁₅ is hydrogen, and R₁₁ is —CH₂C(CH₃)₂CH₂OH, —CH₂CH₂OH, —C(CH₃)₂CH₂OH,—CH(CH₂OH)₂, —CH₂CH(OH)CH₂OH, —CH(CH₃)CH₂OH or

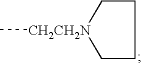

or R₁₅ and R₁₁ are taken together with the nitrogen atom to which they are attached to form a 5-membered heterocyclic ring where R₁₅R₁₁ is represented by —(CH₂)₄—.

Z. The method according to V, wherein the thiazole is characterized by general Formula IIIb:

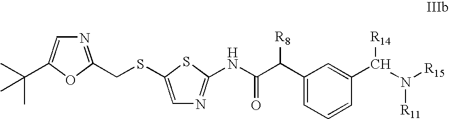

wherein

R₈ and R₁₄ are each independently hydrogen or methyl; and

R₁₅ is hydrogen, and R₁₁ is hydrogen, —CH₂C(CH₃)₂CH₂OH, —CH₂CH₂OH, —(CH₃)₂CH₂OH, —CH(CH₂OH)₂, —CH₂CH(OH)CH₂OH, —CH(CH₃)CH₂OH or

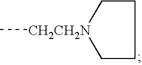

or R₁₅ and R₁₁ are taken together with the nitrogen atom to which they are attached to form a 5-membered heterocyclic ring where R₁₅R₁₁ is represented by —(CH₂)₄—, and enantiomers, diastereomers and pharmaceutically acceptable salts thereof.

AA. The method according to Z, wherein

R₈ and R₁₄ are each independently hydrogen or methyl; and

R₁₅ is hydrogen, and

R₁₁ is —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

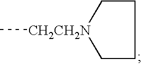

or R₁₅ and R₁₁ are taken together with the nitrogen atom to which they are attached to form a 5-membered heterocyclic ring where R₁₅R₁₁ is represented by —(CH₂)₄—.

AB. The method according to V, wherein the thiazole is characterized by general Formula IIIc:

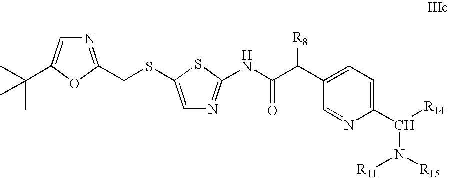

wherein

R₈ and R₁₄ are each independently hydrogen or methyl; and

R₁₅ is hydrogen, and

R₁₁ is hydrogen, —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

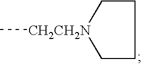

or R₁₅ and R₁₁ are taken together with the nitrogen atom to which they are attached to form a 5-membered heterocyclic ring where R₁₅R₁₁ is represented by —(CH₂)₄—, and enantiomers, diastereomers and pharmaceutically acceptable salts thereof.

AC. The method according to claim AB, wherein

R₈ and R₁₄ are each independently hydrogen or methyl; and

R₁₅ is hydrogen, and

R_{11 is —CH}₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or

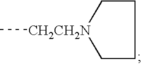

or R₁₅ and R₁₁ are taken together with the nitrogen atom to which they are attached to form a 5-membered heterocyclic ring where R₁₅R₁₁ is represented by —(CH₂)₄—.

AD. The method according to V, wherein the thiazole is selected from the group consisting of:
N-[5-[[[5-(1,1-dimethylethyl)-2-oxazolyl]methyl]thio]-2-thiazolyl]-4-[[[2-hydroxy-1-(hydroxymethyl)ethyl]amino]methyl]benzeneacetamide;
4-[[(2,3-dihydroxypropyl)amino]methyl]-N-[5-[[[5-(1,1-dimethylethyl)-2-oxazolyl]methyl]thio]-2-thiazolyl]benzeneacetamide;
(R)-4-[[(2,3-dihydroxypropyl)amino]methyl]-N-[5-[[[5-(1,1-dimethylethyl)-2-oxazolyl]methyl]thio]-2-thiazolyl]benzeneacetamide;
(S)-4-[[(2,3-dihydroxypropyl)amino]methyl]-N-[5-[[[5-(1,1-dimethylethyl)-2-oxazolyl]methyl]thio]-2-thiazolyl]benzeneacetamide;
4-(aminomethyl)-N-[5-[[[5-(1,1-dimethylethyl)-2-oxazolyl]methyl]thio]-2-thiazolyl]benzeneacetamide;
N-[5-[[[5-(1,1-dimethylethyl)-2-oxazolyl]methyl]thio]-2-thiazolyl]-4-[[(3-hydroxy-2,2-dimethylpropyl)amino]methyl]benzeneacetamide;
N-[5-[[[5-(1,1-dimethylethyl)-2-oxazolyl]methyl]thio]-2-thiazolyl]-4-(1-pyrrolidinylmethyl)benzeneacetamide;
N-[5-[[[5-(1,1-dimethylethyl)-2-oxazolyl]methyl]thio]-2-thiazolyl]-4-[[(2-hydroxyethyl)amino]methyl]benzeneacetamide;
N-[5-[[[5-(1,1-dimethylethyl)-2-oxazolyl]methyl]thio]-2-thiazolyl]-4-[[[2-(1pyrrolidinyl)ethyl]amino]methyl]benzeneacetamide;
N-[5-[[[5-(1,1-dimethylethyl)-2-oxazolyl]methyl]thio]-2-thiazolyl]-4-[[(2-hydroxy-1,1-dimethylethyl)amiono]methyl]benzeneacetamide;
N-[5-[[[5-(1,1-dimethylethyl)-2-oxazolyl]methyl]thio]-2-thiazolyl]-3-[[(2-hydroxyethyl)amino]methyl]benzeneacetamide;
N-[5-[[[5-(1,1-dimethylethyl)-2-oxazolyl]methyl]thio]-2-thiazolyl]-3-[[(3-hydroxy-2,2-dimethylpropyl)amino]methyl]benzeneacetamide;
3-(aminomethyl)-N-[5-[[[5-(1,1-dimethylethyl)-2-oxazolyl]methyl]thio]-2-thiazolyl]benzeneacetamide;
N-[5-[[[5-(1,1-dimethylethyl)-2-oxazolyl]methyl]thio]-2-thiazolyl]-4-[(1-[(2-hydroxy-1-methylethyl)amino]methyl]benzeneacetamide;
N-[5-[[[5-(1,1-dimethylethyl)-2-oxazolyl]methyl]thio]-2-thiazolyl]-4-[1[(2-hydroxyethyl)amino]ethyl]benzeneacetamide;
N-[5-[[[5-(1,1-dimethylethyl)-2-oxazolyl]methyl]thio]-2-thiazolyl]-4-[1-[(2-hydroxy-1-methylethyl)amino]ethyl]benzeneacetamide;
(α)-N-[5-[[[5-(1,1-dimethylethyl)-2-oxazolyl]methyl]thio]-2-thiazolyl]-4-[[(2-hydroxyethyl)amino]methyl]-.alpha.-methylbenzeneacetamide;
N-[5-[[[5-(1,1-dimethylethyl)-2-oxazolyl]methyl]thio]-2-thiazolyl]-6-[[(2-hydroxyethyl)amino]methyl]-3-pyridineacetamide; and
N-[5-[[[5-(1,1-dimethylethyl)-2-oxazolyl]methyl]thio-2-thiazolyl]-4-[[(2-hydroxyethyl)amino]methyl]-α-methylbenzeneacetamide; and
   pharmaceutically acceptable salts thereof.

AE. The method according to any one of A through AD, wherein said pain is selected from the group comprising chronic pain, inflammatory pain and neuropathic pain.

### REFERENCES

Barboric M. et al., NfκB Binds P-TEFb to Stimulate Transcriptional Elongation by RNA Polymerase II. Molecular Cell, 2001, Vol. 8, 327-337.
Besson J. M., The neurobiology of pain. Lancet, 1999, 353(9164), 1610-1615.
Brower, New paths to pain relief. Nat. Biotechnol., 2000, 18(4), 387-391.
Chao S. H. and Price D. H., Flavopiridol inactivates P-TEFb and blocks most RNA polymerase II transcription in vivo. J. Biol. Chem., 2001, 276(34), 31793-9.
Chaplan S R, Bach F W, Pogrel J W, Chung J M, and Yaksh, T L. (1994) Quantitative assessment of tactile allodynia in the rat paw. J. Neurosci. Methods 53: 55-63.
Dai Y. and Grant S., Cyclin-dependent kinase inhibitors. Curr. Opin. Pharmacol, 2003, 3(4), 362-370.
Falco G. D. et al., CDK9, a member of the cdc2-like family of kinases, binds to gp 130, the receptor of the IL-6 family of cytokines. Oncogene, 2002, 21(49), 7464-7470.
Feldmann and Maini, Nat. Med., 2003, 9 (10); 356-61.
Firestein, 2003, Nature 423, 356-361.
Han et al.; 2003, Autoimmunity, 28, 197-208.
Hargreaves, K: Pain 32(1) (January 1988) 77-88.
Huwe et al., Small molecules as inhibitors of cyclin-dependent kinases. Angew. Chem. Int. Ed. Engl., 2003, 42(19), 2122-2138.
Kim et al., Phosphorylation of the RNA polymerase II carboxyl-terminal domain by CDK9 is directly responsible for human immunodeficiency virus type 1 Tat-activated transcriptional elongation. Mol. Cell. Bio.l, 2002, 22(13), 4622-4637.
Koltzenburg M, Neural mechanisms of cutaneous nociceptive pain. Clin. J. Pain, 2000, 16(3 Suppl), 131-138.
Laufer S., Gay S. And Brune K., Inflammation and Rheumatic Diseases—The molecular basis of novel therapies, Thieme, 2003.
Lee K. M. et al., Spinal NfκB activation induces COX-2 upregulation and contributes to inflammatory pain hypersensitivity. European Journal ofNeuroscience, 2004, Vol. 19, 3375-3381.
Liu H. and Herrmann C., Differential Localization and Expression of the CDK9 42 k and 55 k Isoforms. J. Cell Physiol., 2004, 203, 251-260.
MacLachlan T. K. et al., Binding of CDK9 to TRAF2. J. Cell. Biochem., 1998, 71(4), 467-478.
Malmberg A B and Basbaum A I. (1998) Partial sciatic nerve injury in the mouse as a model of neuropathic pain: behavioral and neuroanatomical correlates. Pain 76: 215-2.
Meijer L, Leclerc S., Leost M., Properties and potential applications of chemical inhibitors of cyclin-dependent kinases, Pharmacol. The.r 1999, 82(2-3):279-284.
Sausville, E. A. Trends Molec. Med. 2002, 8, S32-S37.
Tian B. et al., Identification of direct genomic targets downstream of the NfκB transcription factor mediating TNF signaling. JBC, 2005, as manuscript M500437200.
Wang D, et al., Inhibition of human immunodeficiency virus type 1 transcription by chemical cyclin-dependent kinase inhibitors. J. Virol. 2001; 75: 7266-7279.
Watkins L. R. et al., Glial proinflammatory cytokines mediate exaggerated pain states: implications for clinical pain. Adv. Ex.p Med. Biol., 2003, 521, 1-21.
West et al.; 2001, J. Viro.l 75(18), 8524-8537.
Zhou M. et al., Coordination of transcription factor phosphorylation and histone methylation by the P-TEFb Kinase during human immunodeficiency virus type I transcription, J. Viro.l 2004, 78(24):13522-13533.

What is claimed is:

1. A method for the treatment of pain comprising administering to a subject in need thereof an amount of at least one inhibitor of a cyclin-dependent kinase (CDK) sufficient to elicit a hypoalgesic effect.

2. The method according to claim 1, wherein said at least one inhibitor of a cyclin-dependent kinase comprises a thiazole of general Formula I, II or III: 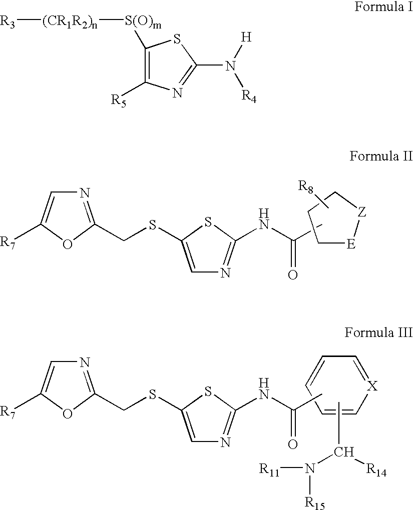
wherein
R₁ and R₂ are independently hydrogen, fluorine or alkyl;
R₃ is aryl or heteroaryl;
R₄ is hydrogen, alkyl, cycloalkyl, aryl, cycloalkylalkyl, arylalkyl, heteroaryl, heteroarylalkyl, heterocycloalkyl, heterocycloalkylalkyl; or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-alkyl, SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkyl-heteroaryl, C(NNO₂)NH-heterocyloalkyl, C(NNO₂)NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHCO-alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₆)NH-alkyl, C(NOR₆)NH-cycloalkyl, C(NOR₆)NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, or C(NOR₆)NH-alkyl-heterocycloalkyl;
R₅ is hydrogen or alkyl;
R₆ is hydrogen, alkyl, cycloalkyl, aryl, cycloalkylalkyl, arylalkyl, heteroaryl, heteroarylalkyl, heterocycloalkyl or heterocycloalkylalkyl;
R₇ is alkyl;
R₈ is hydrogen or alkyl;
R₉ and R₁₀ are each independently hydrogen, alkyl, substituted alkyl, cycloalkyl or substituted cycloalkyl;
R₁₁ is hydrogen or alkyl substituted with one or two hydroxyl groups or with an amino group; R₁₅ is hydrogen or alkyl; or R₁₅ and R₁₁ taken together are —(CH₂)_{q}— where q is 3, 4, 5 or 6;
R₁₄ is hydrogen or alkyl;
X is CH or N;
Z is NR₉ or CHNR₉R₁₀;
m is 0, 1, or 2;
n is 1, 2, or 3;
E is a covalent bond or (CH₂)ₚ; and
p is 1, 2 or 3;
and enantiomers, diastereomers and pharmaceutically acceptable salts thereof.

3. The method according to claim 2, wherein the thiazole is characterized by general Formula I, wherein
R₁ and R₂ are independently hydrogen, fluorine or alkyl; 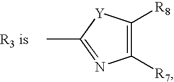
wherein Y is oxygen, sulfur or NR₉;
R₄ is alkyl, cycloalkyl, aryl, cyoloalkylalkyl, arylalkyl, heteroaryl, heteroarylalkyl, heterocycloalkyl, heterocycloalkylalkyl, or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkyl-heteroaryl, C(NNO₂)NH-heterocyloalkyl, C(NNO₂)NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryl, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHC alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₆)NH-alkyl, C(NOR₆)NH-cycloalkyl, C(NOR₆)NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, C(NOR₆)NH-alkyl-heterocycloalkyl;
R₅ is hydrogen or alkyl;
R₆ is hydrogen, alkyl, cycloalkyl, aryl, cycloalkylalkyl, arylalkyl, heteroaryl, heteroarylalkyl, heterocycloalkyl or heterocycloalkylalkyl;
R₇ and R₈ are independently hydrogen, alkyl, substituted alkyl, cycloalkyl, aryl, substituted aryl, cycloalkylalkyl, arylalkyl, heteroaryl, substituted heteroaryl, heteroarylalkyl, heterocycloalkyl, heterocycloalkyalkyl; and
R₉ is hydrogen, alkyl, cycloalkyl, aryl, alkylcycloalkyl, arylalkyl, heteroaryl, heteroarylalkyl, heterocycloalkyl or heterocycloalkylalkyl.

4. The method according to claim 2, wherein the thiazole characterized by general Formula I, wherein
R₁ and R₂ are independently hydrogen, fluorine or alkyl; 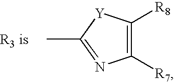
wherein Y is oxygen;
R₄ is CO-alkyl, CO-alkyl-aryl, CO-cycloalkyl, CO-alkyl-heteroaryl, CO-alkyl-heteroalkyl, CO-alkyl-heterocycloalkyl, CONH-alkyl, CONH-alkyl-aryl, CONH-cycloalkyl, or CONH-alkyl-heterocycloalkyl;
R₅ is hydrogen; and
R₇ and R₈ are hydrogen;
m is 0; and
n is 1.

5. The method according to claim 2, wherein the thiazole is characterized by general Formula I, wherein
R₁ and R₂ are independently hydrogen, fluorine or alkyl; 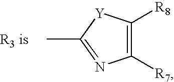
wherein Y is oxygen;
R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;
R₅ is hydrogen;
R₇ and R₈ are alkyl;
m is 0; and
n is 1.

6. The method according to claim 2, wherein the thiazole is characterized by general Formula I, wherein
R₁ and R₂ are independently hydrogen, fluorine or alkyl; 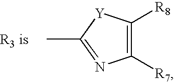
wherein Y is oxygen;
R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;
R₅ is hydrogen;
R₇ is hydrogen;
R₈ is alkyl;
m is 0; and
n is 1.

7. The method according to claim 2, wherein said the thiazole is characterized by general Formula I, wherein
R₁ and R₂ are independently hydrogen, fluorine or alkyl; 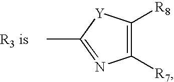
wherein Y is oxygen;
R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;
R₅ is hydrogen;
R₇ is alkyl;
R₈ is hydrogen;
m is 0; and
n is 1.

8. The method according to claim 2, wherein the thiazole is characterized by general Formula I, wherein
R₁ and R₂ are independently hydrogen, fluorine or alkyl; 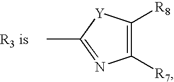
wherein Y is sulfur;
R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;
R₅ is hydrogen;
R₇ is hydrogen;
R₈ is alkyl;
m is 0; and
n is 1.

9. The method according to claim 2, wherein the thiazole is characterized by general Formula I, wherein
R₁ and R₂ are independently hydrogen, fluorine or alkyl; 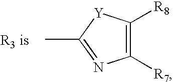
wherein Y is sulfur;
R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;
R₅ is hydrogen;
R₇ is alkyl;
R₈ is hydrogen;
m is 0; and
n is 1.

10. The method according to claim 2, wherein the thiazole is characterized by general Formula I, wherein
R₁ and R₂ are independently hydrogen, fluorine or alkyl; 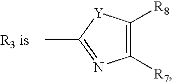
wherein Y is NR₉;
R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;
R₅ is hydrogen;
R₇ is hydrogen;
R₈ is alkyl;
R₉ is hydrogen, alkyl, cycloalkyl, aryl, alkyl-cycloalkyl, alkyl-aryl, heteroaryl, alkyl-heteroaryl, heterocycloalkyl, or alkyl-heterocycloalkyl;
m is 0; and
n is 1.

11. The method according to claim 2, wherein the thiazole is characterized by general Formula I, wherein
R₁ and R₂ are independently hydrogen, fluorine or alkyl; 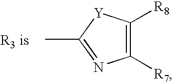
wherein Y is NR₉;
R₄ is CO-alkyl, CO-alkyl-aryl, CO-alkyl-heteroalkyl, CO-cycloalkyl, CO-alkyl-heterocycloalkyl, CO-alkyl-heteroaryl, CONH-alkyl, CONH-alkyl-aryl, CONH-cycloalkyl, or CONH-alkyl-heterocycloalkyl;
R₅ is hydrogen;
R₇ is alkyl;
R₈ is hydrogen;
R₉ is alkyl;
m is 0; and
n is 1.

12. The method according to claim 2, wherein the thiazole is characterized by general Formula I, wherein
R₁ and R₂ are independently hydrogen, fluorine or alkyl; 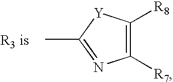
wherein Y is NR₉;
R₄ is CO-alkyl, CO-alkyl-aryl, CO-cycloalkyl, CO-alkyj-heteroaryl, CO-alkyl-heteroalkyl, CO-alkyl-heterocycloalkyl, CONH-alkyl, CONH-alkyl-aryl, CONH-cycloalkyl, or CONH-alkyl-heterocycloalkyl;
R₅ is hydrogen;
R₇ is alkyl;
R₈ is hydrogen;
R₉ is hydrogen;
m is 0; and
n is 1.

13. The method according to claim 2, wherein the thiazole is characterized by general Formula I, and is selected from the group consisting of:
N-[5-[[5-Ethyl-2-oxazolyl)methyl]thio]-2-thiazoly]acetamide;
N-[5-[[5-Ethyl-2-oxazolyl)methyl]thio]-2-thiazolyl]benzamide;
N-[5-[[5-Ethyl-2-oxazolyl)methyl]thio]-2-thiazolyl]benzenesulfonamide;
N-[5-[[(4,5-Dimethyl-2-oxazolyl)methyl]thio]-2-thiazolyl]acetamide;
N-[5-[[(5-t-Butyl-2-oxazolyl)methyl]thio]-2-thiazolyl]acetamide;
N-[5-[[5-t-Butyl-2-oxazolyl)methyl]thio]-2-thiazolyl]trimethylacetamide;
N-[5-[[(4-Ethyl-2-oxazolyl)methyl]thio]-2-thiazolyl]acetamide;
N-[5-[[(5-t-Butyl-2-oxazolyl)methyl]thio]-2-thiazolyl]-N′-cyano-N″-(2,6-difluorophenyl)guanidine;
N-[5-[[(5-Isopropyl-2-oxazolyl)fluoromethyl]thio]-2-thiazolyl]acetamide;
N-[5-[[(5-t-Butyl-2-oxazolyl)methyl]thio]-2-thiazolyl]aminophenyl-4-(2-hydroxyethyl)sulfonamide;
N-[5-[[(5-t-Butyl-2-oxazolyl)methyl]thio]-2-thiazolyl]aminophenyl-4-sulfonamide;
N-[5-[[(5-t-Butyl-2-oxazolyl)methyl]thio]-2-thiazolyl]-4-aminopyrimidine;
N-[5-[[(5-t-Butyl-2-oxazolyl)methyl]thio]-2-thiazolyl]-3-(hydroxymethyl)aniline;
N-[5-[[(5-t-Butyl-2-oxazolyl)methyl]thio]-2-thiazolyl]-2-aminopyridine;
N-[5-[[(5-t-Butyl-2-oxazolyl)methyl]thio]-2-thiazolyl]-2-[5-[(((3-hydroxy-2,2-dimethyl)propyl)amino)methyl]]aminopyridine;
N-[5-[[[5-(1,1-dimethylethyl)-2-oxazolyl]-methyl]thio]-2-thiazolyl]-4-piperidinecarboxamide; and
a pharmaceutically acceptable salt thereof.

14. The method according to claim 2, wherein the thiazole is characterized by general Formula II: 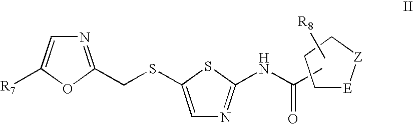
wherein R₇ is alkyl; R₈ is hydrogen or alkyl; Z is NR₉ or CHNR₉R₁₀; and R₉ and R₁₀ are each independently hydrogen, alkyl, substituted alkyl, cycloalkyl or substituted cycloalkyl.

15. The method according to claim 14, wherein the thiazole is characterized by general Formula IIa: 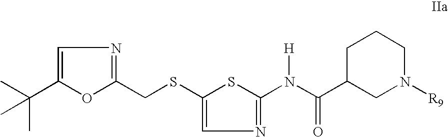
wherein R₉ is hydrogen, alkyl, substituted alkyl or cycloalkyl.

16. The method according to claim 14, wherein the thiazole is characterized by general Formula IIb: 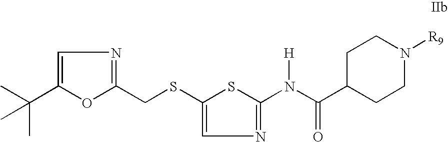
wherein R₉ is alkyl, substituted alkyl or cycloalkyl.

17. The method according to claim 14, wherein the thiazole is characterized by general Formula IIc: 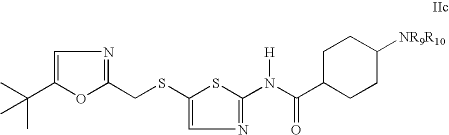
wherein R₉ and R₁₀ are each independently hydrogen, alkyl, substituted alkyl or cycloalkyl with the proviso that both R₉ and R₁₀ are not hydrogen.

18. The method according to claim 14, wherein the thiazole is selected from the group consisting of:
(±)-N-[5-[[[5-(1,1-dimethylethyl)-2-oxazolyl]methyl]thio]-2-thiazolyl]-3-piperidinecarboxamide;
(±)-1-(2,3-dihydroxypropyl)-N-[5-[[[5-(1,1-dimethylethyl)-2-oxazolyl]methyl]thio]-2-thiazolyl]-4-piperidinecarboxamide;
N-[5-[[[5-(1,1-dimethylethyl)-2-oxazolyl]methyl]thio]-2-thiazolyl]-1-(1-methylethyl)-4-piperidinecarboxamide;
1-cyclopropyl-N-[5-[[[5-(1,1-dimethylethyl)-2-oxazolyl]methyl]thio]-2-thiazolyl]-4-piperidinecarboxamide;
N-[5-[[[5-(1,1-dimethylethyl)-2-oxazolyl]methyl]thio]-2-thiazolyl]-1-(2-hydroxyethyl)-4-piperidinecarboxamide;
(R)-N-[5-[[[5-(1,1-dimethylethyl)-2-oxazolyl]methyl]thio]-2-thiazolyl]-3-piperidinecarboxamide;
(S)-N-[5-[[[5-(1,1-dimethylethyl)-2-oxazolyl]methyl]thio]-2-thiazolyl]-3-piperidinecarboxamide;
cis-4-amino-N-[5-[[[5-(1,1-dimethylethyl)-2-oxazolyl]methyl]thio]-2-thiazolyl]cyclohexylcarboxamide; and
trans-4-amino-N-[5-[[[5-(1,1-dimethylethyl)-2-oxazolyl]methyl]thio]-2-thiazolyl]cyclohexylcarboxamide; and
a pharmaceutically acceptable salt thereof.

19. The method according to claim 2, wherein the thiazole is characterized by general Formula III: 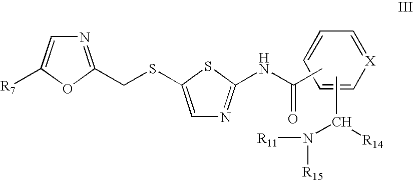
wherein
R₇ is alkyl;
R₈ and R₁₄ are each independently hydrogen or alkyl; and X is CH or N.

20. The method according to claim 19, wherein the thiazole is characterized by general Formula IIIa: 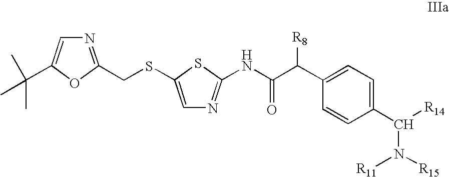
wherein
R₈ and R₁₄ are each independently hydrogen or methyl; and
R₁₅ is hydrogen, and R₁₁ is hydrogen, —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or 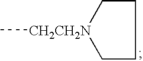
or
R₁₅ and R₁₁ taken together are —(CH₂)₄—.

21. The method according to claim 19, wherein the thiazole is characterized by general Formula IIIb: 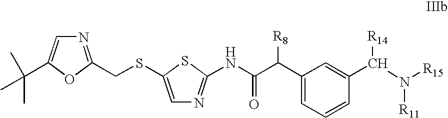
wherein
R₈ and R₁₄ are each independently hydrogen or methyl; and
R₁₅ is hydrogen, and R₁₁ is hydrogen, —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or 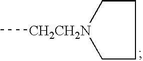
or R₁₅ and R₁₁ taken together are —(CH₂)₄—.

22. The method according to claim 19, wherein the thiazole is characterized by general Formula IIIc: 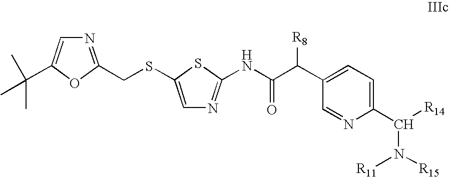
wherein
R₈ and R₁₄ are each independently hydrogen or methyl; and
R₁₅ is hydrogen, and R₁₁ is hydrogen, —CH₂C(CH₃)₂CH₂OH, —CH₂CH₂OH, —C(CH₃)₂CH₂OH, —CH(CH₂OH)₂, —CH₂CH(OH)CH₂OH, —CH(CH₃)CH₂OH or 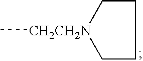
or R₁₅ and R₁₁ taken together are —(CH₂)₄—.

23. The method according to claim 19, wherein the thiazole is selected from the group consisting of:
N-[5-[[[5-(1,1-dimethylethyl)-2-oxazolyl]methyl]thio]-2-thiazolyl]-4-[[[2-hydroxy-1-(hydroxymethyl)ethyl]amino]methyl]benzeneacetamide;
4-[[(2,3-dihydroxypropyl)amino]methyl]-N-[5-[[[5-(1,1-dimethylethyl)-2-oxazolyl]methyl]thio]-2-thiazolyl]benzeneacetamide;
(R)-4-[[(2,3-dihydroxypropyl)amino]methyl]-N-[5-[[[5-(1,1-dimethylethyl)-2-oxazolyl]methyl]thio]-2-thiazolyl]benzeneacetamide;
(S)-4-[[(2,3-dihydroxypropyl)amino]methyl]-N-[5-[[[5-(1,1-dimethylethyl)-2-oxazolyl]methyl]thio]-2-thiazolyl]benzeneacetamide;
4-(aminomethyl)-N-[5-[[[5-(1,1-dimethylethyl)-2-oxazolyl]methyl]thio]-2-thiazolyl]benzeneacetamide;
N-[5-[[[5-(1,1-dimethylethyl)-2-oxazolyl]methyl]thio]-2-thiazolyl]-4-[[(3-hydroxy-2,2-dimethylpropyl)amino]methyl]benzeneacetamide;
N-[5-[[[5-(1,1-dimethylethyl)-2-oxazolyl]methyl]thio]-2-thiazolyl]-4-(1-pyrrolidinylmethyl)benzeneacetamide;
N-[5-[[[5-(1,1-dimethylethyl)-2-oxazolyl]methyl]thio]-2-thiazolyl]-4-[[(2-hydroxyethyl)amino]methyl]benzeneacetamide;
N-[5-[[[5-(1,1-dimethylethyl)-2-oxazolyl]methyl]thio]-2-thiazoly]-4-[[[2-(1pyrrolidinyl)ethyl]amino]methyl]benzeneacetamide;
N-[5-[[[5-(1,1-dimethylethyl)-2-oxazolyl]methyl]thio]-2-thiazolyl]-4-[[(2-hydroxy-1,1-dimethylethyl)amino]methyl]benzeneacetamide;
N-[5-[[[5-(1,1-dimethylethyl)-2-oxazolyl]methyl]thio]-2-thiazolyl]-3-[[(2-hydroxyethyl)amino]methyl]benzeneacetamide;
N-[5-[[[5-(1,1-dimethylethyl)-2-oxazolyl]methyl]thio]-2-thiazolyl]-3-[[(3-hydroxy-2,2-dimethylpropyl)amino]methyl]benzeneacetamide;
3-(aminomethyl)-N-[5-[[[5-(1,1-dimethylethyl)-2-oxazolyl]methyl]thio]-2-thiazolyl]benzeneacetamide;
N-[5-[[[5-(1,1-dimethylethyl)-2-oxazolyl]methyl]thio]-2-thiazolyl]-4-[[(2-hydroxy-1-methylethyl)amino]methyl]benzeneacetamide;
N-[5-[[[5-(1,1-dimethylethyl)-2-oxazolyl]methyl]thio]-2-thiazolyl]-4-[1-[(2-hydroxyethyl)amino]ethyl]benzeneacetamide;
N-[5-[[[5-(1,1-dimethylethyl)-2-oxazolyl]methyl]thio]-2-thiazolyl]-4-[1-[(2-hydroxy-1-methylethyl)amino]ethyl]benzeneacetamide;
(α)-N-[5-[[[5-(1,1-dimethylethyl)-2-oxazolyl]methyl]thio]-2-thiazolyl]-4-[[(2-hydroxyethyl)amino]methyl]-.alpha.-methylbenzeneacetamide;
N-[5-[[[5-(1,1-dimethylethyl)-2-oxazolyl]methyl]thio]-2-thiazolyl]-6-[[(2-hydroxyethyl)amino]methyl]-3-pyridineacetamide; and
N-[5-[[[5-(1,1-dimethylethyl)-2-oxazolyl]methyl]thio]-2-thiazolyl]-4-[[(2-hydroxyethyl)amino]methyl]-α-methylbenzeneacetamide; and
a pharmaceutically acceptable salt thereof.

24. A thiazole selected from the group consisting of:
(±)-N-[5-[[[5-(1,1-dimethylethyl)-2-oxazolyl]methyl]thio]-2-thiazolyl]-3-piperidinecarboxamide;
(R)—N-[5-[[[5-(1,1-dimethylethyl)-2-oxazolyl]methyl]thio]-2-thiazoly]-3-piperidinecarboxamide;
(S)—N-[5-[[[5-(1,1-dimethylethyl)-2-oxazolyl]methyl]thio]-2-thiazoly]-3-piperidinecarboxamide;
cis-4-amino-N-[5-[[[5-(1,1-dimethylethyl)-2-oxazolyl]methyl]thio]-2-thiazolyl]cyclohexylcarboxamide;
trans-4-amino-N-[5-[[[5-(1,1-dimethylethyl)-2-oxazolyl]methyl]thio]-2-thiazolyl]cyclohexylcarboxamide;
N-[5-[[[5-(1,1-Dimethylethyl)-2-oxazolyl]methyl]thio]-2-thiazolyl]-4-[[[2-hydroxy-1-(hydroxymethyl)ethyl]amino]methyl]benzeneacetamide;
N-[5-[[[5-(1,1-Dimethylethyl)-2-oxazolyl]methyl]thio]-2-thiazolyl-4-[[(2-hydroxy-1,1-dimethylethyl)amino]methyl]benzeneacetamide;
4-[[(2,3-Dihydroxypropyl)amino]methyl]-N-[5-[[[5-(1,1-dimethylethyl)-2-oxazolyl]methyl]thio]-2-thiazolyl]benzeneacetamide;
(R)-4-[[(2,3-Dihydroxypropyl)amino]methyl]-N-[5-[[[5-(1,1-dimethylethyl)-2-oxazolyl]methyl]thio]-2-thiazolyl]benzeneacetamide;
(S)-4-[[(2,3-Dihydroxypropyl)amino]methyl]-N-[5-[[[5-(1,1-dimethylethyl)-2-oxazolyl]methyl]thio]-2-thiazolyl]benzeneacetamide; and
a pharmaceutically acceptable salt thereof.

25. The method according to claim 1, wherein said pain is selected from the group comprising chronic pain, inflammatory pain, and neuropathic pain.

26. A pharmaceutical composition comprising at least one inhibitor of a cyclin-dependent kinase, wherein said at least one inhibitor is a thiazole characterized by any one of general Formula I, II or III: 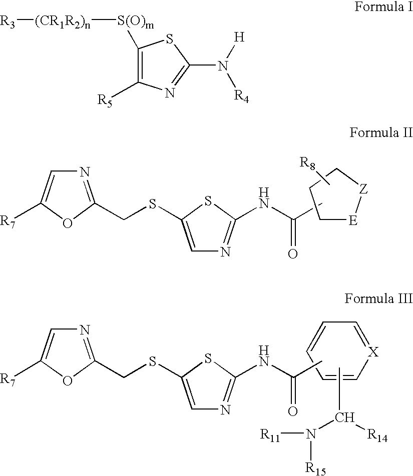
wherein
R₁ and R₂ are independently hydrogen, fluorine or alkyl;
R₃ is aryl or heteroaryl;
R₄ is hydrogen, alkyl, cycloalkyl, aryl, cycloalkylalkyl, arylalkyl, heteroaryl, heteroarylalkyl, heterocycloalkyl, heterocycloalkylalkyl; or CO-alkyl, CO-cycloalkyl, CO-aryl, CO-alkyl-cycloalkyl, CO-alkyl-aryl, CO-heteroaryl, CO-alkyl-heteroaryl, CO-heterocycloalkyl, CO-alkyl-heterocycloalkyl; or CONH-alkyl, CONH-cycloalkyl, CONH-aryl, CONH-alkyl-cycloalkyl, CONH-alkyl-aryl, CONH-heteroaryl, CONH-alkyl-heteroaryl, CONH-heterocycloalkyl, CONH-alkyl-heterocycloalkyl; or COO-alkyl, COO-cycloalkyl, COO-aryl, COO-alkyl-cycloalkyl, COO-alkyl-aryl, COO-heteroaryl, COO-alkyl-heteroaryl, COO-heterocycloalkyl, COO-alkyl-heterocycloalkyl; or SO₂-alkyl, SO₂-cycloalkyl, SO₂-aryl, SO₂-alkyl-cycloalkyl, SO₂-alkyl-aryl, SO₂-heteroaryl, SO₂-alkyl-heteroaryl, SO₂-heterocycloalkyl, SO₂-alkyl-heterocycloalkyl; or C(NCN)NH-alkyl, C(NCN)NH-cycloalkyl, C(NCN)NH-aryl, C(NCNNH)-alkyl-cycloalkyl, C(NCN)NH-alkyl-aryl, C(NCN)NH-heteroaryl, C(NCN)NH-alkyl-heteroaryl, C(NCN)NH-heterocycloalkyl, C(NCN)NH-alkyl-heterocylcoalkyl; or C(NNO₂)NH-alkyl, C(NNO₂)NH-cycloalkyl, C(NNO₂)NH-aryl, C(NNO₂)NH-alkyl-cycloalkyl, C(NNO₂)NH-alkyl-aryl, C(NNO₂)NH-heteroaryl, C(NNO₂)NH-alkyl-heteroaryl, C(NNO₂)NH-heterocyloalkyl, C(NNO₂)NH-alkyl-heterocycloalkyl; or C(NH)NH-alkyl, C(NH)NH-cycloalkyl, C(NH)NH-aryl, C(NH)NH-alkyl-cycloalkyl, C(NH)NH-alkyl-aryI, C(NH)NH-heteroaryl, C(NH)NH-alkyl-heteroaryl, C(NH)NH-heterocycloalkyl, C(NH)NH-alkyl-heterocycloalkyl; or C(NH)NHCO-alkyl, C(NH)NHCO-cycloalkyl, C(NH)NHCO-aryl, C(NH)NHCO-alkyl-cycloalkyl, C(NH)NHCO-alkyl-aryl, C(NH)NHCO-heteroaryl, C(NH)NHCO-alkyl-heteroaryl, C(NH)NHCO-heterocylcloalkyl, C(NH)NHCO-alkyl-heterocycloalkyl; or C(NOR₆)NH-alkyl, C(NOR₆)NH-cycloalkyl, C(NOR₆)NH-aryl, C(NOR₆)NH-alkyl-cycloalkyl, C(NOR₆)NH-alkyl-aryl, C(NOR₆)NH-heteroaryl, C(NOR₆)NH-alkyl-heteroaryl, C(NOR₆)NH-heterocylcoalkyl, or C(NOR₆)NH-alkyl-heterocycloalkyl;
R₅ is hydrogen or alkyl;
R₆ is hydrogen, alkyl, cycloalkyl, aryl, cycloalkylalkyl, arylalkyl, heteroaryl, heteroarylalkyl, heterocycloalkyl or heterocycloalkylalkyl;
R₇ is alkyl;
R₈ is hydrogen or alkyl;
R₉ and R₁₀ are each independently hydrogen, alkyl, substituted alkyl, cycloalkyl or substituted cycloalkyl;
R₁₁ is hydrogen or alkyl substituted with one or two hydroxyl groups or with an amino group; R₁₅ is hydrogen or alkyl; or R₁₅ and R₁₁ taken together are —(CH₂)_{q}— where q is 3, 4, 5 or 6;
R₁₄ is hydrogen or alkyl;
X is CH or N;
Z is NR₉ or CHNR₉R₁₀;
m is 0, 1, or 2;
n is 1, 2, or 3;
E is a covalent bond or (CH₂)ₚ; and
p is 1, 2 or 3;
and enantiomers, diastereomers and pharmaceutically acceptable salts thereof.

27. The pharmaceutical composition according to claim 26, wherein said at least one inhibitor of a cyclin-dependent kinase is N-[5-[[[5-(1,1-dimethylethyl)-2-oxazolyl)methyl]thio]-2-thiazolyl]-4-piperidinecarboxamide and the N-oxide derivatives, prodrug derivatives, protected derivatives, individual isomers, mixtures of isomers, enantiomers, diastereomers, and the pharmaceutically acceptable salts and solvates thereof.
